(19) United States
(12) Patent Application Publication
(10) Pub. No.: US 2005/0153998 A1
(43) Pub. Date: Jul. 14, 2005

(54) Tetrahydroisoquinoline or isochroman compounds

(76) Inventors: Fumitaka Ito, Aichi-ken (JP); Shigeo Hayashi, Aichi-ken (JP); Yoshinobu Hashizume, Aichi-ken (JP); Sachiko Mihara, Aichi-ken (JP)

Correspondence Address: WARNER-LAMBERT COMPANY, 2800 PLYMOUTH RD, ANN ARBOR, MI, 48105, US

(21) Appl. No.: 10/921,632

(22) Filed: Aug. 19, 2004

Related U.S. Application Data
(60) Provisional application No. 60/496,354, filed on Aug. 19, 2003.

(51) Int. Cl. A61K 31/4747; C07D 471/12

(52) U.S. Cl. 514/278; 546/16; 546/17

(57) ABSTRACT

This invention provides the compounds of formula (I): 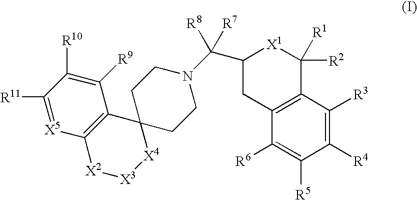
or its a pharmaceutically acceptable ester or amide of such compound, or a pharmaceutically acceptable salt thereof, wherein X¹ is NH; R¹, R², R⁴ through R⁶ and R⁷ through R¹¹ are all hydrogen; R³ is hydroxy; X² and X³ are methylene; X⁴ is a bond; and X⁵ is a carbon atom, and the like. These compounds have ORL1-receptor antagonist activity; and therefore, are useful to treat diseases or conditions such as pain, various CNS diseases etc.

This application is a United States utility application, which claims the benefit of priority to U.S. provisional Application Ser. No. 60/496,354 filed Aug. 19, 2003.

### TECHNICAL FIELD

This invention relates to substituted tetrahydroisoquinoline and isochroman compounds and their pharmaceutically acceptable esters or amides and the pharmaceutically acceptable salts thereof, and a medical use thereof. Also, this invention relates to a pharmaceutical composition comprising one of said compounds or a pharmaceutically acceptable ester or amide, or a pharmaceutically acceptable salt thereof of one of said compounds. The compounds of this invention are useful in treating or preventing disorders or medical conditions selected from pain, CNS disorders and the like. The compounds of this invention having binding affinity for the ORL-1 receptor. In particular, compounds of this invention have selective antagonist activity for said receptor and are useful in treating or preventing disorders or medical conditions which are mediated by overactivation of said receptor.

### BACKGROUND ART

Three types of opioid receptors, μ (mu), δ (delta) and κ (kappa) have been identified. These receptors may be indicated with combinations of OP (abbreviation for Opioid Peptides) and numeric subscripts as suggested by the International Union of Pharmacology (IUPHAR). Namely, OP₁, OP₂ and OP₃ respectively correspond to δ-, κ- and μ-receptors. It has been found out that they belong to G-protein-coupled receptors and distribute in the central nervous system (CNS), peripheries and organs in a mammal. As ligands for the receptors, endogenous and synthetic opioids are known. It is believed that an endogenous opioid peptide produces their effects through an interaction with the major classes of opioid receptors. For example, endorphins have been purified as endogenous opioid peptides and bind to both δ- and μ-receptors. Morphine is a well-known non-peptide opioid analgesic and has binding affinity mainly for μ-receptor. Opiates have been widely used as pharmacological agents, but drugs such as morphine and heroin induce some side effects such as drug addiction and euphoria.

Meunier et al. reported isolation of a seventeen-amino-acid-long peptide from rat brain as an endogenous ligand for an orphan opioid receptor (Nature, Vol. 337, pp. 532-535, Oct. 12, 1995), and said receptor is now known as “opioid receptor-like 1 (abbreviated as ORL-1receptor)”. In the same report, the endogenous opioid ligand has been introduced as agonist for ORL-1 receptor and named as “nociceptine (abbreviated as NC)”. Also, the same ligand was named as “orphanin FQ (abbreviated as OFQ or oFQ)” by Reinscheid et al. (Science, Vol. 270, pp. 792-794, 1995). This receptor may be indicated as OP₄ in line with a recommendation by IUPHAR in 1998 (British Journal of Pharmacology, Vol. 129, pp. 1261-1283, 2000).

Euroceltitique's WO 02/085354 refers to spiropiperidine compounds, but there is no specific disclosure in the publication regarding any compounds having a heteroaryl ring attached directly or through a spacer moiety to the nitrogen atom in the piperidine ring.

### BRIEF DISCLOSURE OF THE INVENTION

The present invention provides a compound of the following formula (I): 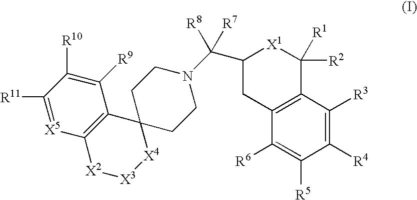
or a pharmaceutically acceptable ester or amide of such compound, or a pharmaceutically acceptable salt thereof, wherein
X¹ represents
   an oxygen atom; or
   N—R¹² wherein R¹² is selected from the group consisting of a hydrogen atom, an alkyl group having 1 to 6 carbon atoms, an alkanoyl group having 1 to 6 carbon atoms, an alkylaminocarbonyl group having 1 to 6 carbon atoms in the alkyl group, an aryl group as defined below, an aryalkyl group having 1 to 6 carbon atoms in the alkyl part and the aryl part as defined below, a heteroaryl group as defined below and a heteroarylalkyl group having 1 to 6 carbon atoms in the alkyl part and heteroaryl part as defined below;
R¹ and R² each independently represent
   a hydrogen atom;
   an alkyl group having 1 to 6 carbon atoms;
   an alkoxy group having 1 to 6 carbon atoms;
   an alkanoyl group having 1 to 6 carbon atoms;
   an alkylcarbonylamino group having 1 to 6 carbon atoms in the alkyl part;
   an alkylaminocarbonyl group having 1 to 6 carbon atoms in the alkyl part;
   a mono-hydroxyalkyl group having 1 to 6 carbon atoms;
   a mono-aminoalkyl having 1 to 6 carbon atoms; or
   an alkoxyalkyl group having 1 to 6 carbon atoms in the alkoxy group and 1 to 6 carbon atoms in the alkyl part; or R¹ and R² taken together form oxo;
R³, R⁴, R⁵ and R⁶ each independently represent
   a hydrogen atom;
   a halogen atom;
   a hydroxy group;
   an alkyl group having 1 to 6 carbon atoms;
   an alkoxy group having 1 to 6 carbon atoms;
   an alkanoyl group having 1 to 6 carbon atoms;
   a mono-hydroxyalkyl group having 1 to 6 carbon atoms;
   a mono-aminoalkyl group having 1 to 6 carbon atoms;
   an alkylcarbonylamino group having 1 to 6 carbon atoms in the alkyl part;
   an alkylaminocarbonyl group having 1 to 6 carbon atoms in the alkyl part;
   an alkylaminosulfonyl group having 1 to 6 carbon atoms in the alkyl part;
   an aryl group as defined below which is linked directly to the benzene ring or is
   attached via a spacer group to the benzene ring, and the spacer group is defined as below; or
   a heteroaryl group as defined below which is linked directly to the benzene ring or is attached via a spacer group to the benzene ring, and the spacer group is defined as below;
   provided that at least one of R³ through R⁶ must represents a hydrogen atom
R⁷ and R⁸ both represent hydrogen atoms or taken together form oxo;
R⁹, R¹⁰ and R¹¹ each independently represent a hydrogen atom, a halogen atom or an alkyl group having 1 to 6 carbon atoms;
X², X³ and X⁴ each independently represent methylene, an oxygen atom, NR¹³, where R¹³ is defined as a hydrogen atom or an alkyl group having from 1 to 6 carbon atoms, or carbonyl, provided that at least one of X², X³ and X⁴ must represent methylene or carbonyl; or
X⁴ represents a bond and X² and X³ each independently represent methylene, an oxygen atom, NR¹³, where R¹³ is defined as a hydrogen atom or an alkyl group having from 1 to 6 carbon atoms, carbonyl, provided that at least one of X² and X³ must be methylene or carbonyl;
wherein the methylene in the definitions of X², X³ and X⁴ is each independently unsubstituted or substituted by at least one alkyl groups having 1 to 6 carbon atoms;
X⁵ represents a —CR¹⁴ or a nitrogen atom wherein R¹⁴ represents a hydrogen atom, a halogen atom or an alkyl group having 1 to 6 carbon atoms;
said amino parts of the alkylcarbonylamino groups and alkylaminocarbonyl groups in the definitions of R¹ through R⁶ and R¹² are unsubstituted or substituted by an alkyl group having 1 to 6 carbon atoms;
said aryl groups and aryl parts of aralkyl groups referred to in the definitions of R¹ through R⁶ and R¹² are aromatic hydrocarbon groups having 5 to 14 carbon atoms;
said heteroaryl groups and heteroaryl parts of the heteroarylalkyl groups referred to in the definitions of R³ through R⁶ and R¹² are 5- to 7-membered heteroaryl groups containing 1 to 3 oxygen, sulfur and/or nitrogen atoms; and
said spacer groups referred to in the definitions of R¹ and R² are each independently selected from the groups consisting of an oxygen atom, sulfonyl and carbonyl.
A preferred compound of this invention is a compound of formula I wherein
X¹ represents
   an oxygen atom; or
   N—R¹² wherein R¹² is selected from the group consisting of a hydrogen atom, an alkyl group having 1 to 6 carbon atoms, an alkanoyl group having 1 to 6 carbon atoms and an alkylaminocarbonyl group having 1 to 6 carbon atoms in the alkyl group;
R¹ and R² each independently represent
   a hydrogen atom;
   an alkyl group having 1 to 6 carbon atoms;
   an alkoxy group having 1 to 6 carbon atoms;
   an alkanoyl group having 1 to 6 carbon atoms;
   an alkylcarbonylamino group having 1 to 6 carbon atoms in the alkyl part;
   an alkylaminocarbonyl group having 1 to 6 carbon atoms in the alkyl part;
   a mono-hydroxyalkyl group having 1 to 6 carbon atoms;
   a mono-aminoalkyl having 1 to 6 carbon atoms; or
   an alkoxyalkyl group having 1 to 6 carbon atoms in the alkoxy group and 1 to 6 carbon atoms in the alkyl part; or R¹ and R² taken together form oxo;
R³, R⁴, R⁵ and R⁶ each independently represent
   a hydrogen atom;
   a halogen atom;
   a hydroxy group;
   an alkyl group having 1 to 6 carbon atoms;
   an alkoxy group having 1 to 6 carbon atoms;
   an alkanoyl group having 1 to 6 carbon atoms;
   a mono-hydroxyalkyl group having 1 to 6 carbon atoms; or
   a mono-aminoalkyl group having 1 to 6 carbon atoms;
   provided that at least one of R³ through R⁶ must represents a hydrogen atom;
R⁷ and R⁸ both represent hydrogen atoms or taken together form oxo;
R⁹, R¹⁰ and R¹¹ each independently represent a hydrogen atom, a halogen atom or an alkyl group having 1 to 6 carbon atoms; X², X³ and X⁴ each independently represent methylene, an oxygen atom, NR¹³, where R¹³ is defined as a hydrogen atom or an alkyl group having from 1 to 6 carbon atoms, or carbonyl, provided that at least one of X², X³ and X⁴ must represent methylene or carbonyl; or
X⁴ represents a bond and X² and X³ each independently represent methylene, an oxygen atom, NR¹³, where R¹³ is defined as a hydrogen atom or an alkyl group having from 1 to 6 carbon atoms, or carbonyl, provided that at least one of X² and X³ must be methylene or carbonyl;
wherein the methylene in the definitions of X², X³ and X⁴ are each independently unsubstituted or substituted by at least one alkyl groups having 1 to 6 carbon atoms;
X⁵ represents a —CR¹⁴ or a nitrogen atom wherein R¹⁴ represents a hydrogen atom, a halogen atom or an alkyl group having 1 to 6 carbon atoms;
said amino parts of the alkylcarbonylamino groups and alkylaminocarbonyl groups in the definitions of R¹ through R⁶ and R¹² are unsubstituted or substituted by an alkyl group having 1 to 6 carbon atoms;
said aryl groups and aryl parts of aralkyl groups referred to in the definitions of R¹ through R⁶ and R¹² are aromatic hydrocarbon groups having 5 to 14 carbon atoms;
said heteroaryl groups and heteroaryl parts of the heteroarylalkyl groups referred to in the definitions of R³ through R⁶ and R¹² are 5- to 7-membered heteroaryl groups containing 1 to 3 oxygen, sulfur and/or nitrogen atoms; and
said spacer groups referred to in the definitions of R¹ and R² are each independently selected from the groups consisting of an oxygen atom, sulfonyl and carbonyl.

A more preferred compound of this invention is a compound of formula I wherein X¹ represents N—R¹² and R¹² represents a hydrogen atom, an alkyl group having 1 to 6 carbon atoms or an alkanoyl group having 1 to 6 carbon atoms.

A more preferred compound of this invention is a compound of formula I wherein X¹ represents an oxygen atom.

A more preferred compound of this invention is a compound of formula I wherein X⁴ represents a bond and X² and X³ each independently represent methylene, an oxygen atom, NR¹³, where R¹³ is defined as a hydrogen atom or an alkyl group having from 1 to 6 carbon atoms, or carbonyl, provided that at least one of X² and X³ must be methylene or carbonyl, wherein the methylene is unsubstituted or is substituted by at least one alkyl group having 1 to 6 carbon atoms.

A more preferred compound of this invention is a compound of formula I wherein X⁵ represents CR¹⁴.

A more preferred compound of this invention is a compound of formula I wherein R⁷ and R⁸ both represent hydrogen atoms.

A more preferred compound of this invention is a compound of formula I wherein R¹ and R² both represent hydrogen atoms or one of R¹ and R² is a hydrogen atom and the other one is an alkyl group having 1 to 6 carbon atoms.

A more preferred compound of this invention is a compound of formula I wherein R¹ and R² taken together form oxo.

A more preferred compound of this invention is a compound of formula I wherein R³ to R⁶ each independently represent a hydrogen atom, a hydroxy group or a halogen atom, provided that at least one of R³ to R⁶ must represent a hydrogen atom.

Suitable compounds according to the present invention are selected from:
1′-(1,2,3,4-tetrahydroisoquinolin-3-ylmethyl)-2,3-dihydrospiro[indene-1,4′-piperidine];
1′-[(2-acetyl-1,2,3,4-tetrahydroisoquinolin-3-yl)methyl]-2,3-dihydrospiro[indene-1,4′-piperidine];
1′-(2-methyl-1,2,3,4-tetrahydroisoquinolin-3-yl)methyl]-2,3-dihydrospiro[indene-1,4′-piperidine];
1′-[(3S)-1,2,3,4-tetrahydroisoquinolin-3-ylmethyl]-2,3-dihydrospiro[indene-1,4′-piperidine];
(3R)-3-(2,3-dihydro-1′H-spiro[indene-1,4′-piperidin]-1′-ylmethyl)-1,2,3,4-tetrahydroisoquinolin-7-ol;
3-(2,3-dihydro-1′H-spiro[indene-1,4′-piperidin]-1′-ylmethyl)-1,2,3,4-tetrahydroisoquinolin-6-ol;
5-bromo-3-(2,3-dihydro-1′H-spiro[indene-1,4′-piperidin]-1′-ylmethyl)-1,2,3,4-tetrahydroisoquinolin-8-ol;
3-(2,3-dihydro-1′H-spiro[indene-1,4′-piperidin]-1′-ylmethyl)-2-methyl-1,2,3,4-tetrahydroisoquinolin-8-ol;
3-(2,3-dihydro-1′H-spiro[indene-1,4′-piperidin]-1′-ylmethyl)-1,2,3,4-tetrahydroisoquinolin-8-ol;
3-(2,3-dihydro-1′H-spiro[indene-1,4′-piperidin]-1′-ylmethyl)-5-fluoro-1,2,3,4-tetrahydroisoquinolin-8-ol
5-fluoro-3-[(1-methyl-1,2-dihydro-1′H-spiro[indole-3,4′-piperidin]-1′-yl)methyl]-1,2,3,4-tetrahydroisoquinolin-8-ol;
5-chloro-3-(2,3-dihydro-1′H-spiro[indene-1,4′-piperidin]-11′-ylmethyl)-1,2,3,4-tetrahydroisoquinolin-8-ol;
5-chloro-3-(1′H,3H-spiro[2-benzofuran-1,4′-piperidin]-1′-ylmethyl)-1,2,3,4-tetrahydroisoquinolin-8-ol;
5-chloro-3-[(1-methyl-1,2-dihydro-1′H-spiro[indole-3,4′-piperidin]-1′-yl)methyl]-1,2,3,4-tetrahydroisoquinolin-8-ol;
1′-{[(3S)-1-methyl-1,2,3,4-tetrahydroisoquinolin-3-yl]methyl}-2,3-dihydrospiro[indene-1,4′-piperidine];
3-[(3,3-dimethyl-1′H,3H-spiro[2-benzofuran-1,4′-piperidin]-1′-yl)methyl]-1,2,3,4-tetrahydroisoquinolin-8-ol;
3-[(1′-methyl-1′,2′-dihydro-1H-spiro[piperidine-4,3′-pyrrolo[2,3-b]pyridin]-1-yl)methyl]-1,2,3,4-tetrahydroisoquinolin-8-ol;
3-[(6-fluoro-1′H,3H-spiro[2-benzofuran-1,4′-piperidin]-1′-yl)methyl]-1,2,3,4-tetrahydroisoquinolin-8-ol;
3-[(5-fluoro-1-methyl-1,2-dihydro-1′H-spiro[indole-3,4′-piperidin]-1′-yl)methyl]-1,2,3,4-tetrahydroisoquinolin-8-ol;
1′-[(8-hydroxy-1,2,3,4-tetrahydroisoquinolin-3-yl)methyl]-1-methylspiro[indole-3,4′-piperidin]-2(1H)-one;
1′-[(5-chloro-8-hydroxy-1,2,3,4-tetrahydroisoquinolin-3-yl)methyl]-1-methylspiro[indole-3,4′-piperidin]-2(1H)-one;
1′-[(5-fluoro-8-hydroxy-1,2,3,4-tetrahydroisoquinolin-3-yl)methyl]-1-methylspiro[indole-3,4′-piperidin]-2(1H)-one;
5-fluoro-3-[(6-fluoro-1′H,3H-spiro[2-benzofuran-1,4′-piperidin]-1′-yl)methyl]-1,2,3,4-tetrahydroisoquinolin-8-ol;
5-chloro-3-[(6-fluoro-1′H,3H-spiro[2-benzofuran-1,4′-piperidin]-1′-yl)methyl]-1,2,3,4-tetrahydroisoquinolin-8-ol;
5-chloro-3-[(5-fluoro-1-methyl-1,2-dihydro-1′H-spiro[indole-3,4′-piperidin]-1′-yl)methyl]-1,2,3,4-tetrahydroisoquinolin-8-ol;
5-fluoro-3-[(5-fluoro-1-methyl-1,2-dihydro-1′H-spiro[indole-3,4′-piperidin]-1′-yl)methyl]-1,2,3,4-tetrahydroisoquinolin-8-ol;
1′-[(5-chloro-8-hydroxy-1,2,3,4-tetrahydroisoquinolin-3-yl)methyl]-5-fluoro-1-methylspiro[indole-3,4′-piperidin]-2(1H)-one;
1′-[(5-chloro-1,2,3,4-tetrahydroisoquinolin-3-yl)methyl]-1-methyl-1,2-dihydrospiro[indole-3,4′-piperidine] or
5-chloro-3-(2,3-dihydro-1′H-spiro[indene-1,4′-piperidin]-1′-ylmethyl)-3,4-dihydroisoquinolin-1(2H)-one or a pharmaceutically acceptable salt thereof.

Another group of suitable compounds of this invention includes 3-[(5-fluoro-1-methyl-1,2-dihydro-1′H-spiro[indole-3,4′-piperidin]-1′-yl)methyl]-8-hydroxy-3,4-dihydro-1H-isochromen-1-one; and 3-(2,3-dihydro-1′H-spiro[indene-1,4′-piperidin]-1′-ylmethyl)-3,4-dihydro-1H-isochromen-8-ol or a pharmaceutically salt thereof.

Thus, as a further aspect, the present invention provides a pharmaceutical composition comprising a compound of this invention, a pharmaceutically acceptable ester or amide of said compound, or a pharmaceutically acceptable salt thereof with a pharmaceutically acceptable excipient, diluent or carrier.

A further aspect of this invention is to provide a pharmaceutical composition for the treatment of disease conditions caused by overactivation of ORL1-receptor, in a mammalian subject, which comprises a therapeutically effective amount of the compound of this invention, a pharmaceutically acceptable ester or amide of said compound, or a pharmaceutically acceptable salt thereof.

A further aspect of this invention is to provide a pharmaceutical composition comprising a therapeutically effective amount of a compound of this invention, a pharmaceutically acceptable ester or amide of said compound, or a pharmaceutically acceptable salt thereof, wherein the disease or condition is pain, sleep disorders, eating disorders including anorexia and bulimia; anxiety and stress conditions; immune system diseases; locomotor disorder; memory loss, cognitive disorders and dementia including senile dementia, Alzheimer's disease, Parkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders including hypotension, bradycardia and stroke; renal disorders including water excretion, sodium ion excretion and syndrome of inappropriate secretion of antidiuretic hormone (SIADH); gastrointestinal disoders; airway disorders including adult respiratory distress syndrome (ARDS); autonomic disorders including suppression of micturition reflex; metabolic disorders including obesity; cirrhosis with ascites; sexual dysfunctions; altered pulmonary function including obstructive pulmonary disease; and tolerance to or dependency on a narcotic analgesic., preferably pain, sleep disorders, eating disorders including anorexia and bulimia; stress conditions; memory loss, cognitive disorders, gastrointestinal disorders; sexual dysfunctions; tolerance to or dependency on a narcotic analgesic.

Another aspect of this invention relates to use of a compound of this invention, a pharmaceutically acceptable ester or amide of said compound, a pharmaceutically acceptable salt thereof, or a pharmaceutical composition comprising said compound, ester, amide or salt, as a medicament.

A further aspect of this invention relates to use of a compound of this invention, a pharmaceutically acceptable ester or amide of said compound, a pharmaceutically acceptable salt thereof, or a pharmaceutical composition comprising said compound, ester, amide or salt in the manufacture of a medicament to treat a disease or condition for which an ORL1-receptor antagonist is indicated.

A further aspect of this invention relates to use of a compound of this invention, a pharmaceutically acceptable ester or amide of said compound, a pharmaceutically acceptable salt thereof, or a pharmaceutical composition comprising said compound, aster, amide or salt for manufacture of a medicament to treat a disease or condition referred to above.

This invention also relates to a method of treating a disease or condition for which an ORL1-receptor antagonist is indicated, in a mammal, including a human being, comprising administering to a mammal requiring such treatment an effective amount of a compound of this invention, a pharmaceutically acceptable ester or amide of said compound, a pharmaceutically acceptable salt thereof, or a pharmaceutical composition comprising said compound, ester, amide or salt.

This invention further relates to a method of treating a disease or condition referred to above, in a mammal, including a human, comprising administering to a mammal requiring such treatment an effective amount of a compound of this invention, a pharmaceutically acceptable ester or amide of said compound, or a pharmaceutically acceptable salt thereof, or a pharmaceutical composition comprising said compound, ester, amide or salt,.

Thus, the compounds of the present invention are useful for the general treatment of pain, particularly neuropathic pain. Physiological pain is an important protective mechanism designed to warn of danger from potentially injurious stimuli from the external environment. The system operates through a specific set of primary sensory neurons and is exclusively activated by noxious stimuli via peripheral transducing mechanisms (Millan 1999 Prog. Neurobio. 57: 1-164 for an integrative Review). These sensory fibres are known as nociceptors and are characterized by small diameter axons with slow conduction velocities. Nociceptors encode the intensity, duration and quality of noxious stimulus and by virtue of their topographically organized projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumor related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other types of pain include but are not limited to:
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis;
Central pain or ‘thalamic pain’ as defined by pain caused by lesion or dysfunction of the nervous system including but not limited to central post-stroke pain, multiple sclerosis, spinal cord injury, Parkinson's disease and epilepsy;
Heart and vascular pain including but not limited to angina, myocardical infarction, mitral stenosis, pericarditis, Raynaud's phenomenon, scleredoma, scleredoma, skeletal muscle ischemia;
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis;
Head pain including but not limited to migraine, migraine with aura, migraine without aura cluster headache, tension-type headache; and
Orofacial pain including but not limited to dental pain, temporomandibular myofascial pain.

### DETAILED DESCRIPTION OF THE INVENTION

As used herein, the term “halogen” means fluoro, chloro, bromo and iodo, preferably fluoro or chloro.

As used herein, the term “alkyl” means straight or branched chain saturated radicals, including, but not limited to methyl, ethyl, n-propyl, isopropyl, n-butyl, iso-butyl, secondary-butyl, tertiary-butyl.

As used herein, the term “alkoxy” means alkyl-O—, including, but not limited to methoxy, ethoxy, n-propoxy, isopropoxy, n-butoxy, iso-butoxy, secondary-butoxy, tertiary-butoxy.

As used herein, the term “alkanoyl” means a group having carbonyl such as R′-C(O)— wherein R′ is H, C₁₋₅ alkyl, phenyl or C₃₋₆ cycloalkyl, including, but not limited to formyl, acetyl, ethyl-C(O)—, n-propyl-C(O)—, isopropyl-C(O)—, n-butyl-C(O)—, iso-butyl-C(O)—, secondary-butyl-C(O)—, tertiary-butyl-C(O)—, cyclopropyl-C(O)—, cyclobutyl-C(O)—, cyclopentyl-C(O)—, cyclohexyl-C(O)—, and the like.

As used herein, the term “aryl” means a monocyclic or bicyclic aromatic carbocyclic ring of 5 to 14 carbon atoms including, but not limited to, phenyl, naphthyl, preferably phenyl.

The term “heteroaryl” means a 5- to 7-membered aromatic hetero mono-cyclic ring comprising either (a) 1 to 4 nitrogen atoms or (b) one oxygen or one sulphur atom and 0 to 2 nitrogen atoms including, but not limited to, pyrazolyl, furyl, thienyl, oxazolyl, tetrazolyl, thiazolyl, imidazolyl, thiadiazolyl, pyridyl, pyrimidinyl, pyrrolyl, thiophenyl, pyrazinyl, pyridazinyl, isooxazolyl, isothiazolyl, triazolyl, furazanyl, and the like.

The term “haloalkyl”, as used herein, means an alkyl radical which is substituted by halogen atoms as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl groups and the like.

The term “haloalkoxy”, as used herein, means haloalkyl-O—, including, but not limited to, fluoromethoxy, difluoromethoxy, trifluoromethoxy, 2-fluoroethoxy, 2,2-difluoroethoxy, 2,2,2-trifluoroethoxy, 2,2,2-trichloroethoxy, 3-fluoropropoxy, 4-fluorobutoxy, chloromethoxy, trichloromethoxy, iodomethoxy and bromomethoxy groups and the like.

Where the compounds of formula (I) contain hydroxy groups, they may form esters. Examples of such esters include esters with a hydroxy group and esters with a carboxy group. The ester residue may be an ordinary protecting group or a protecting group which can be cleaved in vivo by a biological method such as hydrolysis.

The term “protecting group” means a group, which can be cleaved by a chemical method such as hydrogenolysis, hydrolysis, electrolysis or photolysis.

Compounds of formula I of this invention having free amino or hydroxy groups can be converted into prodrugs. This invention also encompasses these prodrugs including the esters and amides that are more specifically described as below. These prodrugs also include compounds wherein an amino acid residue, or a polypeptide chain of two or more (e.g., two, three or four) amino acid residues is covalently joined through an amide or ester bond to a free amino or hydroxy group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed in this invention. For instance, free amine groups can be derivatized as amides. The amide moieties may incorporate groups including but not limited to amine and carboxylic acid functionalities. Free hydroxy groups may be derivatized using groups including but not limited to hemisuccinates, phosphate esters, dimethylaminoacetates, and phosphoryloxymethyloxycarbonyls, as outlined in D. Fleisher, R. Bong, B. H. Stewart, Advanced Drug Delivery Reviews (1996) 19, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39, 10.

The term “ester” or “amide” means a protecting group which can be cleaved in vivo by a biological method such as hydrolysis and forms a free acid or a free amine, or salt thereof. Whether a compound is such a derivative or not can be determined by administering it by intravenous injection to an experimental animal, such as a rat or mouse, and then studying the body fluids of the animal to determine whether or not the compound or a pharmaceutically acceptable salt thereof can be detected.

Preferred examples of groups for forming an ester with a hydroxy group and for forming an amide with a amino group include: lower aliphatic alkanoyl groups, for example: alkanoyl groups, such as the formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl and henicosanoyl groups; halogenated alkylcarbonyl groups, such as the chloroacetyl, dichloroacetyl, trichloroacetyl, and trifluoroacetyl groups; alkoxyalkylcarbonyl groups, such as the methoxyacetyl group; and unsaturated alkylcarbonyl groups, such as the acryloyl, propioloyl, methacryloyl, crotonoyl, isocrotonoyl and (E)-2-methyl-2-butenoyl groups; more preferably, the lower aliphatic alkanoyl groups having from 1 to 6 carbon atoms; aromatic alkanoyl groups, for example: arylcarbonyl groups, such as the benzoyl, α-naphthoyl and β-naphthoyl groups; halogenated arylcarbonyl groups, such as the 2-bromobenzoyl and 4-chlorobenzoyol groups; lower alkylated arylcarbonyl groups, such as the 2, 4,6-trimethylbenzoyl and 4-toluoyl groups; lower alkoxylated arylcarbonyl groups, such as the 4-anisoyl group; nitrated arylcarbonyl groups, such as the 4-nitrobenzoyl and 2-nitrobenzoyl groups; lower alkoxycarbonylated arylcarbonyl groups, such as the 2-(methoxycarbonyl)benzoyl group; and arylated arylcarbonyl groups, such as the 4-phenylbenzoyl group; alkoxycarbonyl groups, for example: lower alkoxycarbonyl groups, such as the methoxycarbonyl, ethoxycarbonyl, propoxycarbonyl, butoxycarbonyl, sec-butoxycarbonyl, t-butoxycarbonyl and isobutoxycarbonyl groups; and halogen- or tri(lower alkyl)silyl-substituted lower alkoxycarbonyl groups, such as the 2,2,2-trichloroethoxycarbonyl and 2-trimethylsilylethoxycarbonyl groups; tetrahydropyranyl or tetrahydrothiopyranyl groups, such as: tetrahydropyran-2-yl, 3-bromotetrahydropyran-2-yl, 4-methoxytetrahydropyran-4-yl, tetrahydrothiopyran-2-yl, and 4-methoxytetrahydrothiopyran-4-yl groups; tetrahydrofuranyl or tetrahydrothiofuranyl groups, such as: tetrahydrofuran-2-yl and tetrahydrothiofuran-2-yl groups; silyl groups, for example: tri(lower alkyl)silyl groups, such as the trimethylsilyl, triethylsilyl, isopropyldimethylsilyl, t-butyldimethylsilyl, methyldiisopropylsilyl, methyldi-t-butylsilyl and triisopropylsilyl groups; and tri(lower alkyl)silyl groups substituted by 1 or 2 aryl groups, such as the diphenylmethylsilyl, diphenylbutylsilyl, diphenylisopropylsilyl and phenyldiisopropylsilyl groups; alkoxymethyl groups, for example: lower alkoxymethyl groups, such as the methoxymethyl, 1,1-dimethyl-1-methoxymethyl, ethoxymethyl, propoxymethyl, isopropoxymethyl, butoxymethyl and t-butoxymethyl groups; lower alkoxylated lower alkoxymethyl groups, such as the 2-methoxyethoxymethyl group; and halo(lower alkoxy)methyl groups, such as the 2,2,2-trichloroethoxymethyl and bis(2-chloroethoxy)methyl groups; substituted ethyl groups, for example: lower alkoxylated ethyl groups, such as the 1-ethoxyethyl and 1-(isopropoxy)ethyl groups; and halogenated ethyl groups, such as the 2,2,2-trichloroethyl group; aralkyl groups, for example: lower alkyl groups substituted by from 1 to 3 aryl groups, such as the benzyl, α-naphthylmethyl, β-naphthylmethyl, diphenylmethyl, triphenylmethyl, α-naphthyldiphenylmethyl and 9-anthrylmethyl groups; and lower alkyl groups substituted by from 1 to 3 substituted aryl groups, where one or more of the aryl groups is substituted by one or more lower alkyl, lower alkoxy, nitro, halogen or cyano substituents, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; alkenyloxycarbonyl groups: such as the vinyloxycarbonyl and aryloxycarbonyl groups; and aralkyloxycarbonyl groups in which the aryl ring may be substituted by 1 or 2 lower alkoxy or nitro groups: such as the benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl groups.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

General Synthesis:

The compounds of formula I of the present invention may be prepared according to known preparation methods, or general procedures or preparation methods illustrated in the following reaction Schemes. Unless otherwise indicated R¹ through R¹² and X¹ through X⁵ in the reaction Schemes and discussion that follow are defined as above. Unless otherwise indicated, reactions in this specification may be carried out at about ambient pressure (i.e., 760 mmHg) and about room temperature (i.e., 25° C.). The reactions in the preparation processes described in this specification may be carried out in suitable solvents. In case of a reaction at a low temperature, methylene chloride may be added to the reaction mixture. In case of a reaction at a high temperature, dimethylformamide may be added to the reaction mixture.

Typical preparation procedures for compounds of formula I of the present invention are as follow:

Protecting Groups:

Amino, hydroxy, mercapto or the like may be protected with a protecting group, and the protecting group may be subsequently removed in an appropriate reaction step according to a known procedure (e.g., Protective Groups in Organic Synthesis edited by T. W. Greene et al. (John Wiely & Sons, 1991)). For example, a primary or a secondary amine may be typically protected by reaction with benzyl chloride and K₂CO₃, and the benzyl group (abbreviated as Bn) may be removed by catalytic hydrogenation over palladium-carbon. Introduction of t-butoxycarbonyl (abbreviated as Boc) to amino group may be carried out using (Boc)₂O under basic condition, and the protecting group may be removed in HCl/EtOAc or HCl/methanol. Hydroxy may be protected with t-butyldimethylsilyl (abbreviated as TBS or TBDMS) in alkylation using NaH. The protecting group may be introduced with TBDMSCI and imidazole in DMF and removed using an appropriate reagent such as tetrabutylammonium fluoride.

Halogenations:

Carboxylic acids or alcohols may be converted to alkyl or acyl halides using halogenation reagents. Conversions of alcohols or carboxylic acids respectively to alkyl halides or acyl halides may be typically carried out using SOCl₂, PCl₅, PCl₃, POCl₃, HBr, PBr₃, HI or the like. A tertiary amine may be added to the reaction mixture.

Formation of Sulfonates:

An alcohol may react with methanesulfonyl chloride or p-toluenesulfonyl chloride using a base to form the corresponding sulfonate respectively known as mesylate or tosylate.

Alkylations of Amines:

Generally, alkylation of amines may be carried out with or without a base. An alkylating agent used in this reaction may have a leaving group such as halo, mesylate, tosylate or triflate. Suitable bases used in this reaction include a tertiary amine (e.g., triethylamine, diisopropylethylamine and pyridine), potassium carbonate, sodium hydride, potassium tert-butoxide, potassium bis(trimethylsilyl)amide and the like. Suitable solvents used in this reaction include acetonitrile, acetone, dichloroethane, chloroform, methylene chloride, tetrahydrofuran (THF), ethyl ether, dimethoxyethane (DME), dimethylformamide (DMF) and the like.

Aminations:

Aminations of alkanols or alkyl halides may be carried out by reactions with cyclic imide compounds such as N-phthalimides followed by hydrazinolysis or hydrolysis. If required, the reactions with phthalimides may be carried out using organophosphorous reagents with or without azo compounds.

Amidations:

If appropriate, a base such as triethylamine, or a base catalysis such as N,N-dimethylaminopyridine (DMAP), 4-pyrrolidinopyridine (PPY) or the like may be employed in this reaction. Suitable solvents for this reaction include hexane, dichloromethane, THF, pyridine and the like.

Amidation-1-Acylation of Amines by Acyl Halides:

Acyl halides may be treated with ammonia or amines for the preparation of amides. This reaction may be carried out and in the presence or absence of an aqueous alkali which may capture the liberated halide ion and controlled by cooling or dilution. Instead of the aqueous alkali, a tertiary amine base such as triethylamine, diisopropylethylamine or pyridine, preferably triethylamine may be used in this reaction. This reaction may be carried out in a reaction inert solvent at a temperature from about 0° C. to about 50° C., preferably about ambient temperature. Acyl halide may also be reacted with arylamines, hydrazine or hydroxylamine under the similar conditions. Amino protections using carbobenzoxy group (abbreviated as Cbz) or t-butoxycarbonyl group (abbreviated as Boc) may be carried out in this way.

Amidation 2-Acylation of Amines by Anhydride:

This reaction may be carried out with ammonia or primary or secondary amines according to a similar procedure described in Amidation 1 above. Ammonia and primary amines may give imides including cyclic imides, wherein two acyl groups are attached to the nitrogen.

Amidation 3-Acylation of Amines by Carboxylic acids:

Carboxylic acids may be treated with ammonia or amine compounds to give amides. This amidation may be carried out in the presence of a coupling agent with or without an additional base at about room temperature. A coupling agent such as dicyclohexylcarbodiimide (DCC) used in a peptide synthesis may be applied to the amidations. Other suitable coupling agents used in these amidations include N,N′-carbonyldiimidazole (CDI), diisopropylcarbodiimide (DIPC), 1-ethyl-3-(3-dimethylaminopropyl)carbodiimide, hydrochloride (WSC, water soluble carbodiimide), benzotriazole-1-yloxy-tris(dimethylamino)phosphonium hexafluorophosphate (BOP) and diphenylphosphorylazide (DPPA) and the like. A cyclic amine may be acylated according to a method analogous to these amidations. If amines are subjected to this reaction in its halogen salt forms, additional amines may be used for trapping hydrogen halides formed.

Amidation 4-Acylation of Amines by Carboxylic Esters:

Carboxylic esters may be converted to unsubstituted, N-substituted or N,N-disubstituted amides. This reaction may be carried out in the presence of a strong base catalysis as well as catalysis by cyanide ion under a high pressure. Hydrazides and hydroxamic acids may be prepared from carboxylic esters with hydrazine and hydroxylamine respectively under similar reaction conditions.

Amidation 5-Acylation of Amines by Amides or Other Acid Derivatives:

A salt of an amine may be subjected to this reaction. In this reaction, NH₂ usually acts as a leaving group. Secondary and primary amines (in the form of their salts) are the most common reagents in this reaction. Acid derivatives, which may be converted to amides, include thiol acids, thiol ethers, acyloxyboranes, 1,1,1-trihalo ketones, α-keto nitrils, acyl azides and the like.

These amidations may be carried out in a reaction inert solvent such as dichloromethane (CH₂Cl₂), alcohols such as methanol, ethanol or butanol (BuOH), acetonitrile, tetrahydrofuran (THF), dimethylfolmamide (DMF), or pyridine or a combination thereof, at a temperature from about −110° C. to the reflux temperature of a solvent or to 300° C. in sealed-tube or under high pressure, for from about 5 minutes to 48 hours.

Hydrolysis of Esters:

Hydrolysis of esters may be carried out in the presence of an acid, base, metal ion, enzyme or nucleophile according to a method known to those skilled in the art. The hydrolysis of esters may be carried out in a reaction inert solvent at a temperature from about 0° C. to the reflux temperature of the solvent for from about 1 to 24 hours. Suitable solvents for the reactions include alcohols such as methanol, ethanol, tetrahydrofuran, acetic acid and the like.

Esterifications:

Carboxylic acids and alcohols afford esters using acid catalysis. Typical catalysis for this reaction include conc. HCl, anhydrous sulfuric acid, p-toluenesulfonic acid and the like. The alcohol generally servers as the solvent, but other reaction inert solvent such as toluene or xylene may be used. The alcohol may be used in large excess, and the water from the reaction mixture may be removed.

Reductions:

Reductions may be carried out using reducing agents. Typical reducing agents are lithium aluminum hydride (LAH), aluminum hydride, AlH₃, Red-Al (i.e., sodium bis(2-methoxy)aluminum hydride), lithium triethylborohydride (LiEt₃BH), a complex formed from lithium trimethoxyaluminum hydride (LiAlH(OMe)₃) and CuI, diisobutylaluminium hydride (DIBAL) and lithium bis(trimethylsilyl)amide (LHMDS), LAH and AlCl₃. Typical milder reducing agents are NaBH₄ and the like in a dipoler aprotic solvent such as Me₂SO, DMF or sulfolane. Zinc with acid or base, SnCl₂, chromium(II) ion and the like are also useful for reductions. Other examples of reduction conditions include use of a borane-tetrahydrofuran complex, a borane-methyl sulfide complex (BH₃-Me₂S), or diisobutylaluminum hydride in an aprotic solvent such as tetrahydrofuran or diethyl ether at a temperature of about −110° C. to about reflux temperature of a solvent.

These reactions may be used for modifying compounds obtained or used in the following synthetic methods.

Scheme 1 illustrates synthetic methods for preparing a compound of formula (I) through each through amidation comprising Steps 1 to 3 and reductive alkylation comprising Steps 4 to 6:

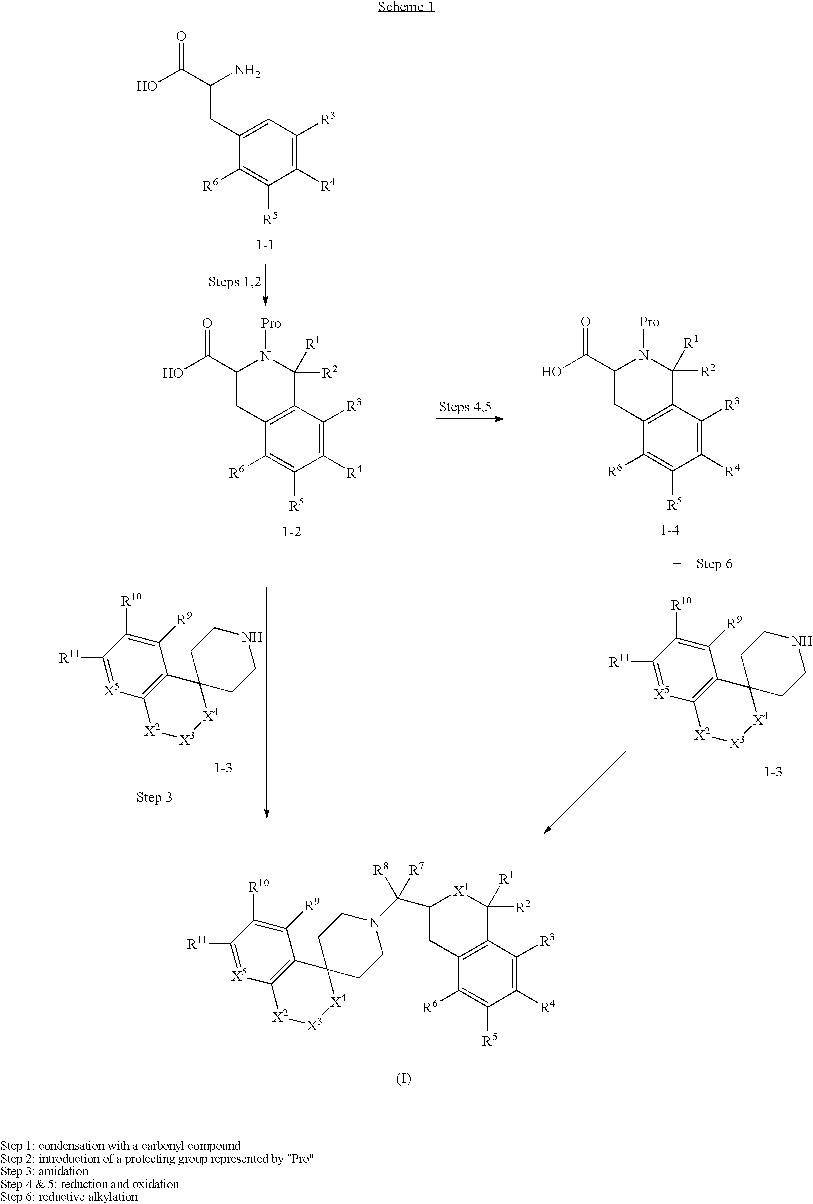

Step 1: This step describes condensation of a compound of formula 1-1 with a carbonyl compound such as HCHO to form the corresponding tetrahydroisoquinoline compound. This reaction may be carried out under conditions of Pictet-Spengler reaction (see for example W. N. Whaley, T. R. Govindachari, Org. React. 6, 151 (1951); R. A. Abramovitch, I. D. Spenser, Advan. Heterocyclic Chem. 3, 79 (1964); K. Stuart, R. Woo-Ming, Heterocycles 3, 223 (1975); D. Soerens et al., J. Org. Chem. 44, 535 (1979); H. Ernest et al., Ber. 114, 1894 (1981); E. Dominguez et al., Tetrahedron 43, 1943 (1987); and M. D. Rozwadowski, Heterocycles 39, 903-931 (1994)). In case of condensation of a compound of formula I with HCHO may be carried out in water at a temperature from about 5° C. to about reflux temperature of a solvent for from about 5 minutes to 4 days, or in case ether R¹ or R² is not hydrogen, −110° C. to about reflux temperature of a solvent.

Step 2: This step describes introduction of a protecting group to the nitrogen atom in the tetrahydroisoquinoline ring of the compound obtained in Step 1 to afford the compound of formula 1-2. Typical protecting groups used in this step are such as “Boc” or “Bn” that are explained in this specification.

Step 3: This step describes reaction of the compound of formula 1-2 obtained in Step 2 with a compound of formula 1-3 to yield the compound of formula (I). This reaction may be carried out under amidation conditions explained in this specification. Typically, this reaction may be carried out using a coupling agent such as WSC in the presence or absence of a base such as triethylamine (Et₃N) in a reaction inert solvent such as alkanol, DMF or the like at a temperature from about −110° C. to about reflux temperature of the solvent for from about 1 minutes to 4 days.

An alternative route for preparing a compound of formula (I) comprises modification of a compound of formula 1-2 to the corresponding aldehyde and reductive alkylation of the aldehyde with a compound of formula 1-3 followed by removal of the protecting group to yield the compound of formula (I). This preparation process is also illustrated in Scheme 1, and Steps 4 through 6 in the preparation process are described as follows.

Steps 4 and 5: A compound of formula 1-2 may be subjected to reduction followed by oxidation to give the corresponding aldehyde compound of formula 1-4. The reduction and oxidation may be carried out by methods known to those skilled in the art, especially by those methods described in this specification. The reduction may be carried out using BH₃-tetrahydrofuran complex in a reaction inert solvent such as THF at a temperature from about 0° C. to room temperature for from about 1 to 60 hours. The oxidation may also be carried out using SO₃-pyridine complex in the presence of a base such as triethylamine. This reaction may be carried out in a reaction inert solvent such as DMSO at a temperature from about 0° C. to room temperature for from about 1 minute to 10 hours.

Step 6: The aldehyde compound obtained in Steps 4 and 5 may be subjected to reductive alkylation with a compound of formula 1-3 followed by removal of the protecting group to yield the compound of formula (I). This reductive alkylation may be carried out under conditions known to those skilled in the art. For example, this reaction may be carried out using NaBH(OAc)₃ in a reaction inert solvent such as dichloromethane at about 0° C. to room temperature for 1 minute to 24 hours.

Compounds of formula I wherein R⁷ and R⁸ together form oxo may be reduced to the corresponding compounds of formula I wherein R⁷ and R⁸ are both hydrogen. This reduction may be carried out using LAH in an aprotic solvent such as THF at a temperature from about −100° C. to the reflux temperature of the solvent used for from about 1 minute to 2 days. This reaction may be also carried out using borane-methyl sulfide instead of LAH.

The compounds of formula 1-1 may be readily prepared from commercially available or known compounds by known methods reported in such as J. Med. Chem. 29, 1302 (1986); Synth. Commun. 17, 1421 (1987); Bull. Chem. Soc. Jpn. 46, 37 (1973); J. Med. Chem. 14, 226 (1971); Tetrahedron asymmetry 3, 555 (1992); J. Med. Chem. 41, 1034 (1998); J. Fluoro. Chem. 70, 39 (1995); and J. Org. Chem. 61, 6974 (1996).

The compounds of formula 1-3 are commercially available or may be prepared from commercially available or known compounds by known methods.

Scheme 2 illustrates preparation methods of the compounds of formula I by alkylation of amine.

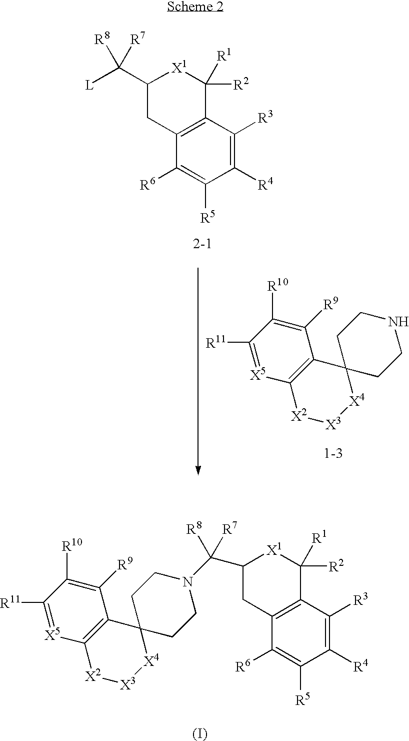

A compound of formula 2-1, wherein L is a leaving group such as chloro, bromo or iodo, may be subjected to N-alkylation with a compound of formula 1-3 to yield the compound of formula I. This reaction may be carried out with a base such as triethylamine, diisopropylethylamine or pyridine in a reaction inert solvent such as DMF, THF at a temperature from about −100° C. to the reflux temperature of the solvent used for from about 1 minute to about 2 days.

The compounds of formula 1-3 are commercially available or readily prepared by known synthetic methods. Scheme 3 exemplifies such preparation methods.

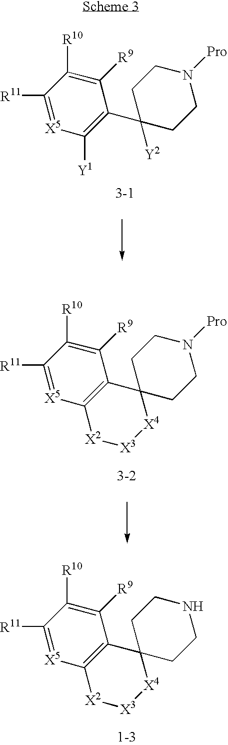

A compound of formula 3-1 may be subjected to an intramolecular cyclization to give the corresponding compound of formula 3-2 and subsequently subjected to deprotection to give the compound of formula 1-3. In the formula 3-1, “Pro” represents a protecting group and “Y¹” and “Y²” respectively represent appropriate substituents that can run an appropriate reaction followed by deprotection to yield the desired compound of formula 1-3. For example, a compound of formula 3-1 wherein Y¹ is halo and Y² is cyano may be subjected to reduction to give the compound of formula 1-3 wherein X² is a nitrogen atom, X³ is CH₂ and X⁴ is a bond. Another example is that a compound of formula 3-1 wherein Y¹ is —CH₂—OH and Y² is OH may be cyclized to give the corresponding compound of the formula 3-2 by removal of water using an water-eliminating agents. Such water-eliminating agents include acetic anhydride, inorganic acid halides, such as thionyl chloride, phosphorus(III) or phosphorus(V) halides, such as phosphorus trichloride or phosphorus pentachloride, phosgene, p-toluenesulfonyl chloride or propanephosphonic anhydride.

In case of indoline compounds represented by formula 1-3, these compounds may also be synthesized according to methods reported in Tetrahedron, Vol. 53, No. 32, pp 10983-10992 or Org. Prep. Proceed. Int.; EN; 27; 6; 1995; 691-694.

The starting materials in the aforementioned general syntheses are commercially available or may be obtained by conventional methods known to those skilled in the art.

The compounds of formula (I), and the intermediates above-mentioned preparation methods can be isolated and purified by conventional procedures, such as recrystallization or chromatographic purification.

The present invention includes salt forms of a compound of the invention as obtained above.

A compound of the invention can be converted from its free base form to a pharmaceutically acceptable salt form by conventional methods known in the art. For example, the formation of the mesylate is a typical procedure and it is carried out as follows. The free base of a compound of the invention is dissolved with methanesulfonic acid in IPA upon heating, and the solution is filtered. The filtrate was cooled and the resulting solids were collected to yield the mesylate as either crystals or a solid.

Insofar as the compounds of this invention are basic compounds, they are capable of forming a wide variety of different salts with various inorganic and organic acids.

The acids which are used to prepare the pharmaceutically acceptable acid addition salts of the base compounds of this invention of formula (I) are those which form non-toxic acid addition salts, i.e., salts containing pharmaceutically acceptable anions, such as the chloride, bromide, iodide, nitrate, sulfate or bisulfate, phosphate or acid phosphate, acetate, lactate, citrate or acid citrate, tartrate or bi-tartrate, succinate, malate, fumarate, gluconate, saccharate, benzoate, methanesulfonate, ethanesulfonate, benzenesulfonate, p-toluenesulfonate and pamoate (i.e., 1.1′-methylene-bis-(2-hydroxy-3-naphthoate). The acid addition salts can be prepared by conventional procedures.

For administration to human patients, the total daily dose of the compounds of this invention is typically in the range of 0.01 mg to 3000 mg depending, of course, on the mode of administration. For example, oral administration may require a total daily dose of from 0.01 mg to 1000 mg. The total dose may be administered in single or divided dosages. These dosages are based on an average human subject having a weight of about 65 to 70 kg. The physician will readily be able to determine doses for subjects whose weight falls outside this range, such as infants and the elderly.

The subject invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assay. Tritiated, i.e., ³H, and carbon-14, i.e., ¹⁴C, isotopes are particularly preferred for their ease of presentation and detectability. Further, substitution with heavier isotopes such as deutrium, i.e., 2H, can afford therapeutic advantage resulting from greater metabolic stability, for example increased in vivo half-life or reduced dosage requirement and, hence, may be preferred in some circumstances. Isotopically labelled compounds of formula (I) of this invention and prodrugs thereof can generally be prepared by carrying out the procedure disclosed in above-disclosed Schemes and/or Examples and Preparations below, by submitting a readily available isotopically labelled reagent for a non-isotopically labeled reagent.

### Method for Assessing Biological Activities:

The compounds of Formula (I) have been found to possess selective affinity for ORL-1receptors and ORL-1 receptor antagonist activity. Thus, these compounds are useful as an analgesic, anti-inflammatory, diuretic, anesthetic, neuroprotective, anti-hypertensive and anti-anxiety agent, and the like, in mammalian subjects, especially humans in need of such agents. The affinity, antagonist activities and analgesic activity can be demonstrated by the following tests respectively.

Affinity for ORL1-Receptors:

ORL1-Receptor Binding Assay:

The human ORL1 receptor transfected HEK-293 cell membranes were incubated for 45 min at 22° C. with 0.4 nM [³H]nociceptin, 1.0 mg of wheat germ agglutinin-coated SPA beads and various concentrations of test compounds in a final volume of 200 μl of 50 mM HEPES buffer pH7.4 containing 10 mM MgCl₂ and 1 mM EDTA. Non-specific binding was determined by the addition of 1 μM unlabeled nociceptin. After the reaction, the assay plate was centrifuged at 1,000 rpm for 1 min and then the radioactivity was measured by a Liquid Scintillation Counter.

The compound prepared in the working example 11 and 22 as described below were tested by this method, and showed a IC₅₀ value of 7.4 nM and 4.9 nM with regard to binding activity for the ORL1 receptor. In this test, the compounds of the present invention exhibited excellent binding activity for the ORL1 receptor.

μ-Receptor Binding Assay:

The human Mu receptor transfected CHO-K1 cell membranes (PerkinElmer) were incubated for 45 min at room temperature with 1.0 nM[³H]DAMGO, 1.0 mg of WGA-coated SPA beads and various concentrations of test compounds in a final volume of 200 μl of 50 mM Tris-HCl buffer pH 7.4 containing 5 mM MgCl₂. NSB was determined by the addition of 1 μM unlabeled DAMGO. After the reaction, the assay plate was centrifuged at 1,000 rpm for 1 min and then the radioactivity was measured by WALLAC 1450 MicroBata Trilux.

Each percent NSB thus obtained was graphed as a function of compound concentration. A sigmoidal curve was used to determine 50% bindings (i.e., IC₅₀ values).

In this testing, the preferred compounds prepared in the working examples appearing hereafter demonstrated higher binding affinity for ORL1-receptors than for mu-receptors.

IC₅₀ (ORL1-receptors) nM/IC₅₀ (mu-receptors) nM<1.0

ORL1 Receptor Functional Assay:

The human ORL1 receptor transfected HEK-293 cell membranes were incubated with 400 pM [³⁵S]GTPγS, 10 nM nociceptin and various concentrations of test compounds in assay buffer (20 mM HEPES, 100 mM NaCl, 5 mM MgCl₂, 1 mM EDTA, 5 μM GDP, 1 mM DTT, pH 7.4) containing 1.5 mg of WGA-coated SPA beads for 90 min at room temperature in a final volume of 200 μL. Basal binding was assessed in the absence of nociceptin and NSB was defined by the addition of unlabelled 10 μM GTPγS. Membrane-bound radioactivity was detected by a Wallac 1450 MicroBeta liquid scintillation counter.

Analgesic Tests:

Tail Flick Test in Mice:

The latency time to withdrawal of the tail from radiant heat stimulation is recorded before and after administration of test compounds. Cut-off time is set to 8 sec.

Acetic Acid Writhing Test in Mice:

Acetic acid saline solution of 0.7% (v/v) is injected intraperitoneally (0.16 ml/10 g body weight) to mice. Test compounds are administered before acetic acid injection. As soon as acetic acid injection, animals are placed in a 1 liter beaker and writhing is recorded for 15 min.

Formalin Licking Test in Mice:

Formalin-induced hind paw licking is initiated by a 20 micro liters subcutaneous injection of a 2% formaline solution into a hind paw of mice. Test compounds are administered prior to formalin injection. Total licking time is recorded for 45 min after formalin injection.

Carrageenan-Induced Mechanical Hyperalgesia Test in Rats:

The response to mechanical nociceptive stimulus is measured using an algesiometer (Ugo Basile, Italy). The pressure is loaded to the paw until rats withdrawal the hind paw. Lambda-Carrageenan saline solution of 1% (w/v) is injected subcutaneously into the hind paw and the withdrawal response is measured before and after the injection. Test compounds are administered at appropriate time point.

Carrageenan-Induced Thermal Hyperalgesia Test in Rats:

The response to thermal nociceptive stimulus is measured using an plantar test apparatus (Ugo Basile, Italy). The radiant heat stimuli is applied to the paw until rats withdrawal the hind paw. Lambda-Carrageenan saline solution of 2% (w/v) is injected subcutaneously into the hind paw and the withdrawal response is measured before and after the injection. This testing method is described in K. Hargreaves, et al., Pain 32: 77-88, 1988.

Chronic Contriction Injury Model (CCI Model):

Chronic contriction injury is made according to Bennett's method (Bennett and Xie, Pain 33: 87-107, 1988). Tactile allodynia in rats is assessed using the von Frey hairs (Stoelting, Ill.) before and after administration with test compounds.

Partial Sciatic Nerve Ligation Model (PSL):

This test may be conducted according to similar procedures described by Z. Seltzer, et al., Pain, 43 (1990) 205-218 (Title: A novel behavioral model of neuropathic pain disorders produced in rats by partial sciatic nerve injury).

Dofetilide Binding Assay

Cell paste of HEK-293 cells expressing the HERG product can be suspended in 10-fold volume of ice-cold wash buffer (50 mM Tris base, 10 mM KCl, 1 mM MgCl₂, adjusted pH 7.4). The cells can be homogenized using a Polytron homogenizer and centrifuged at 48,000 g for 20 minutes at 4° C. The pellet can be resuspended, homogenized and centrifuged once more in the same manner. The resultant supernatant can be discarded and the final pellet can beresuspended (10-fold volume of ice-cold wash buffer) and homogenized. The membrane homogenate can be aliquoted and stored at −80° C. until use. All the manipulation can be done on ice, and stock solution and equipment can be kept on ice at all time.

For the saturation assay, experiments can be conducted in a total volume of 1 ml in 48-well blocks and 200 μl in 96-well plates by Brandel and Skatron method,. respectively. In Brandel method, receptor saturation can be determined by incubating 100 μl of [³H]-dofetilide and 750 μl of HERG homogenate (25-35 μg protein/tube) for 60 minutes at 22° C. in incubation buffer (50 mM Tris base, 10 mM KCl, 1 mM MgCl₂, adjusted pH 7.4). In Skatron method, it can be determined by incubating 20 μl of [³H]-dofetilide and 160 μl of HERG homogenate (25-35 μg protein/well) for 60 minutes at 22° C. in incubation buffer. Total and non-specific bindings (in the presence of 10 μM dofetilide) can be determined in duplicate in a range of [³H]-dofetilide concentrations (1 nM to 50 nM).

For the competition assay, 96-well plates can be used, and a final assay volume can be 200 μl. Various concentrations of test compounds can be incubated in duplicate with 5 nM [³H]-dofetilide (20 μl) and 25-35 μg protein of HERG homogenate (160 μl) for 90 minutes at 22° C. in the incubation buffer. Nonspecific binding can be determined by 10 μM dofetilide (20 μl). The saturation derived K_{D} gained in saturation assay can be used for all Ki calculations.

All incubations can be terminated by rapid vacuum filtration over 0.2% polyethyleneimine soaked glass fibre filter paper using a Brandel cell harvester followed by three washes with ice-cold filtration buffer (50 mM Tris base, 10 mM KCl, 1 mM MgCl₂, adjusted pH 7.4), or using Skatron harvester with the same wash buffer. Receptor-bound radioactivity can be quantified by liquid scintillation counting using Packard LS counter. Competition assays can be performed by counting Wallac GF/B filters on Betaplate scintillation counter (Wallac).

The compounds of the invention may be administered in combination, separately, simultaneously or sequentially, with one or more other pharmacologically active agents. Suitable agents, particularly for the treatment of pain, include:
(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii)barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv)benzodiazepines having a sedative action, e.g. chlordiazepoxide, clorazepate, diazepam, flurazepam, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi)miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) alpha-2-delta ligands, e.g. gabapentin and pregabalin;
(ix)alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl) quinazoline;
(x) tricyclic antidepressants, e.g. desipramine, imipramine, amytriptiline and nortriptiline;
(xi) anticonvulsants, e.g. carbamazepine and valproate;
(xii) serotonin reuptake inhibitors, e.g. fluoxetine, paroxetine, citalopram and sertraline;
(xiii) mixed serotonin-noradrenaline reuptake inhibitors, e.g. milnacipran, venlafaxine and duloxetine;
(xiv) noradrenaline reuptake inhibitors, e.g. reboxetine;
(xv) Tachykinin (NK) antagonists, particularly Nk-3, NK-2 and NK-1 antagonists, e.g. (αR,9R)-7-[3,5-bis(trifluoromethyl)benzyl]-8,9,10,11-tetrahydro-9-methyl-5-(4-methylphenyl)-7H-[1,4]diazocino[2, 1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xvi) Muscarinic antagonists, e.g. oxybutin, tolterodine, propiverine, tropsium chloride and darifenacin;
(xvii) COX-2 inhibitors, e.g. celecoxib, rofecoxib and valdecoxib;
(xviii) Non-selective COX inhibitors (preferably with GI protection), e.g. nitroflurbiprofen (HCT-1026);
(xix) coal-tar analgesics, in particular, paracetamol;
(xx) neuroleptics, such as droperidol;
(xxi) Vanilloid receptor agonists, e.g. resinferatoxin;
(xxii) Beta-adrenergic compounds such as propranolol;
(xxiii) Local anaesthetics, such as mexiletine;
(xxiv) Corticosteriods, such as dexamethasone
(xxv) serotonin receptor agonists and antagonists;
(xxvi) cholinergic (nicotinic) analgesics; and
(xxvii) miscellaneous analgesic agents, such as Tramadol®.

Thus, the invention further provides a combination comprising a compound of the invention or a pharmaceutically acceptable salt, solvate or pro-drug thereof, and a compound or class of compounds selected from the group (i)-(xxvii), above. There is also provided a pharmaceutical composition composition comprising such a combination, together with a pharmaceutically acceptable excipient, diluent or carrier, particularly for the treatment of a disease for which an alpha-2-delta ligand is implicated.

Combinations of the compounds of the present invention and other therapeutic agents may be administered separately, sequentially or simultaneously. Thus, the present invention extends to a kit comprising a compound of the invention, one or more other therapeutic agents, such as those listed above, and a suitable container.

The compounds of the present invention may be formulated by any convenient means using well-known carriers and excipients. Thus, the present invention also provides a pharmaceutical composition comprising a compound of the invention or a pharmaceutically acceptable ester or a pharmaceutically acceptable salt thereof with one or more pharmaceutically acceptable carriers.

For oral administration, tablets containing various excipients such as microcrystalline cellulose, sodium citrate, calcium carbonate, dipotassium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH>8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art. Additionally, it is also possible to administer the compounds of the present invention topically when treating inflammatory conditions of the skin and this may preferably be done by way of creams, jellies, gels, pastes, ointments and the like, in accordance with standard pharmaceutical practice.

### EXAMPLES

The invention is illustrated in the following non-limiting examples in which, unless stated otherwise: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (tlc) and reaction times are given for illustration only; melting points (m.p.) given are uncorrected (polymorphism may result in different melting points); the structure and purity of all isolated compounds were assured by at least one of the following techniques: tlc (Merck silica gel 60 F₂₅₄ precoated TLC plates or Merck NH₂ F₂₅₄ₛ precoated HPTLC plates), mass spectrometry, nuclear magnetic resonance (NMR), infrared red absorption spectra (IR) or microanalysis. Yields are given for illustrative purposes only. Flash column chromatography was carried out using Merck silica gel 60 (230-400 mesh ASTM) or Fuji Silysia Chromatorex® DU3050 (Amino Type, 30˜50 μm). Low-resolution mass spectral data (EI) were obtained on Automass 120 (JEOL) or Integrity (Waters) mass spectrometer. Low-resolution mass spectral data (ESI) were obtained on a Quattro II (Micromass) mass spectrometer. Melting point was obtained using Seiko Instruments Inc. Exstar 6000. NMR data was determined at 270 MHz (JEOL JNM-LA 270 spectrometer) or 300 MHz (JEOL JNM-LA300) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br.=broad, etc. IR spectra were measured by a Shimazu infrared spectrometer (IR-470). Optical rotations were measured using a JASCO DIP-370 Digital Polarimeter (Japan Spectroscopic CO, Ltd.).

Chemical symbols have their usual meanings; b.p. (boiling point), m.p. (melting point), l (liter(s)), ml (milliliter(s)), g (gram(s)), mg(milligram(s)), mol (moles), mmol (millimoles), eq. (equivalent(s)).

### Example 1

### 1′-(1,2,3,4-Tetrahydroisoquinolin-3-ylmethyl)-2,3-dihydrospiro[indene-1,4′-piperidine]

### (A) tert-Butyl 3-(2,3-dihydro-1′H-spiro[indene-1,4′-piperidin]-1′-ylcarbonyl)-3,4-dihydroisoquinoline-2(1H)-carboxylate

To a stirred solution of 2-(tert-butoxycarbonyl)-1,2,3,4-tetrahydroisoquinoline-3-carboxylic acid (610.1 mg, 2.2 mmol, this was prepared according to the reported method by S. E. Gibson et al, Bioorg. Med. ChemLett., 1997, 7, 1289), 2,3-dihydrospiro[1H-indene-1,4′-piperidine] hydrochloride (492.2 mg, 2.2 mmol), triethylamine (0.307 mL, 2.2 mmol), and hydroxybenzotriazole (327 mg, 2.42 mmol) in DMF (15 mL) and THF (10 mL) was added WSC (463.9 mg, 2.42 mmol) at −20° C. After 2 days stirring at room temperature, the reaction mixture was poured into aqueous NaHCO₃ solution (200 mL) and extracted with ether (100 mL×2). The extracts combined were washed with water (70 mL), dried (MgSO₄), filtered, and concentrated. The crude product was purified by silica gel column chromatography (n-hexane/ethyl acetate: 2/1) to give 785.8 mg (80%) of title compound as white solid.

¹H NMR (300 MHz, CDCl₃) δ 7.25-7.04 (8H, m), 5.47-5.27 and 5.08-4.73 (total 2H, each m), 4.65-4.35 and 4.10-3.90 (total 3H, each m), 3.40-2.70 (6H, m), 2.18-2.02 (2H, m), 2.00-1.40 (13H, m, including 9H, s at 1.49 ppm).

### (B) 1′-(1,2,3,4-Tetrahydroisoquinolin-3-ylmethyl)-2,3-dihydrospiro[indene-1,4′-piperidine]

A mixture of tert-butyl 3-(2,3-dihydro-1′H-spiro[indene-1,4′-piperidin]-1′-ylcarbonyl)-3,4-dihydroisoquinoline-2(1H)-carboxylate (154.2 mg, 0.345 mmol) and 10% HCl solution in methanol (15 mL) was stirred at room temperature for 16 h. After evaporation of the solvent, the residue was dissolved in dichloromethane (30 mL) and aqueous NaHCO₃ solution (30 mL). The organic layer was separated and aqueous layer was extracted with dichloromethane (30 mL×3). The extracts combined were washed with brine, dried (MgSO₄), filtered, and concentrated to give 120.8 mg of crude amide product.

¹H NMR (270 MHz, CDCl₃) δ 7.26-7.00 (8H, m), 4.74-4.59 (1H, m), 4.14 (2H, br.s), 4.04-3.90 (2H, m), 3.42-3.20 (1H, m), 3.12-2.78 (5H, m, including 2H, t, J=7.8 Hz at 2.95 ppm), 2.33 (1H, br.s), 2.12 (2H, t, J=7.2 Hz), 2.00-1.73 (2H, m), 1.71-1.54 (2H, m).

To a stirred solution of the above amide product (111.9 mg, 0.323 mmol) in THF (3 mL) was added LiAlH₄ (36.8 mg, 0.969 mmol) at 0° C. After 30 min stirring at 0° C. and room temperature for 16 h, the reaction mixture was quenched with ethyl acetate (20 mL) at 0° C. After 15 min stirring, water (15 m]L) was added to the reaction mixture and stirring was continued another 30 min. The organic layer was separated and the aqueous layer was extracted with ethyl acetate (20 mL×3). The extracts combined were dried (MgSO₄), filtered, and concentrated. The crude product was purified by preparative TLC (silica gel plate: CH₂Cl₂/methanol/25% NH₄OH: 150/10/1) to afford 85 mg (79.1%) of title compound as pale brownish white solid.

¹H NMR (300 MHz, CDCl₃) δ 7.25-7.02 (8H, m), 4.10 (2H, s), 3.13-3.03 (1H, m), 2.99-2.79 (4H, m, including 2H, t, J=7.3 Hz at 2.90 ppm), 2.72 (1H, 1H, dd, J=3.8, 16.1 Hz), 2.62-2.52 (1H, dd, J=9.0, 12.5 Hz), 2.44 (1H, dd, J=4.4, 13.7 Hz), 2.36 (1H, ddd, J=2.6, 12.1, 12.3 Hz), 2.30 (1H, br.s), 2.18-2.08 (1H, m), 2.05-1.87 (4H, m, including 2H, t, J=7.3 Hz at 2.02 ppm), 1.59-1.49 (2H, m).

This solid (80.8 mg, 0.243 mmol) and citric acid (46.7 mg, 0.243 mmol) was dissolved in mixed solvent (30 mL of methanol and 30 mL of CH₂Cl₂), and the solution was stirred at room temperature for 2 h. The solvent was evaporated and resulting residue was solidified from CH₂Cl₂/n-hexane and collected by filtration to give 113.3 mg of citrate salt.

MS(ESI positive) m/z: 333.18(M+H)⁺.

IR(KBr): 3400, 1717, 1589, 1456, 1440, 1393, 1209, 758 cm⁻¹

Anal. Calcd for C₂₃H₂₈N₂—C₆H₈O₇-2H₂O: C, 62.13; H, 7.19; N, 5.00. Found: C, 62.76; H, 7.03; N, 4.86.

### Example 2

### 1′-[(2-Acetyl-1,2,3,4-tetrahydroisoquinolin-3-yl)methyl]-2,3-dihydrospiro[indene-1,4′-piperidine]

To a stirred solution of 1′-(1,2,3,4-tetrahydroisoquinolin-3-ylmethyl)-2,3-dihydrospiro[indene-1,4′-piperidine] (71.7 mg, 0.216 mmol, prepared in Example 1) and triethylamine (0.0753 mL, 0.54 mmol) in CH₂Cl₂ (2 mL) was added acetyl chloride (0.0154 mL, 0.216 mmol) at room temperature and the resulting reaction mixture was refluxed with stirring for 3 h. The reaction mixture was quenched with water (20 mL) and extracted with CH₂Cl₂ (25 mL×3). The extracts combined were washed with brine, dried (MgSO₄), filtered, and concentrated. The residue was purified by preparative TLC (silica gel plate: CH₂Cl₂/methanol:20/1) to afford 74.8 mg (92.5%) of title compound.

¹H NMR (300 MHz, CDCl₃) δ 7.25-7.08 (8H, m), 5.25-5.15 (0.4H, m), 5.14 (0.6H, d, J=17.8 Hz), 4.64 (0.4H, d, J=16.3 Hz), 4.49 (0.4H, d, J=16.1 Hz), 4.34-4.20 (0.6H, m), 4.25 (0.6H, d, J=18.3 Hz), 3.13-2.64 (6H, m), 2.43-2.34 (1H, m), 2.32-2.09 (6H, m, including 1.2H, s at 2.26 ppm and 0.8H, s at 2.21 ppm), 2.02-1.78 (4H, m), 1.53-1.43 (2H, m).

This was dissolved in CH₂Cl₂ and converted to HCl salt by treating with HCl solution in methanol followed by concentration, collection, and dry to afford 69.2 mg of HCl salt.

MS(ESI positive) m/z: 375.22(M+H)⁺.

Anal. Calcd for C₂₅H₃₀N₂O—HCl-2H₂O: C, 67.17; H, 7.89; N, 6.27. Found: C, 67.26; H, 7.47; N, 6.27.

### Example 3

### 1′-[(2-Methyl-1,2,3,4-tetrahydroisoquinolin-3-yl)methyl]-2,3-dihydrospiro[indene-1,4′-piperidine]

To a stirred solution of tert-butyl 3-(2,3-dihydro-1′H-spiro[indene-1,4′-piperidin]-1′-ylcarbonyl)-3,4-dihydroisoquinoline-2(1H)-carboxylate (179 mg, 0.4 mmol, prepared in Example 1 (A)) in THF (5 mL) was added LiAlH₄ (68.3 mg, 1.8 mmol) at room temperature. After 16 h stirring at room temperature, the reaction mixture was quenched with ethyl acetate (10 mL) at 0° C. After 30 min stirring, water (15 mL) was added to the reaction mixture and stirring was continued another 30 min. The organic layer was separated and the aqueous layer was extracted with ethyl acetate (15 mL×3). The extracts combined were dried (MgSO₄), filtered, and concentrated. The crude product was purified by preparative TLC (silica gel plate: CH₂Cl₂/methanol: 10/1) to afford 61.2 mg (44.2%) of title compound.

¹H NMR (270 MHz, CDCl₃) δ 7.25-7.09 (7H, m), 7.06-7.00 (1H, m), 3.84 (1H, d, J=15.8 Hz), 3.74 (1H, d, J=16.0 Hz), 3.03-2.71 (7H, m, including 2H, t, J=7.2 Hz at 2.89 ppm), 2.64 (1H, dd, J=4.9, 12.4 Hz), 2.48 (3H, s), 2.37-2.10 (3H, m), 2.05-1.87 (4H, m, including 2H, t, J=7.6 Hz at 2.01 ppm), 1.57-1.47 (2H, m).

This was converted to HCl salt similar to that described in Example 2 to afford 9.5 mg of HCl salt.

MS (ESI positive) m/z: 347 (M+H)⁺.

### Example 4

### 1-[(3S)-1,2,3,4-Tetrahydroisoquinolin-3-ylmethyl]-2,3-dihydrospiro[indene-1,4′-piperidine]

This was prepared according to the procedure described in Example 1 using (3S)-2-(tert-butoxycarbonyl)-1,2,3,4-tetrahydroisoquinoline-3-carboxylic acid instead of 2-(tert-butoxycarbonyl)-1,2,3,4-tetrahydroisoquinoline-3-carboxylic acid except for the reduction step. In this case borane-methyl sulfide complex instead of LiAlH₄ was used for the reduction. Thus the mixture of the amide product (1.60 g, 4.61 mmol) and borane-methyl sulfide complex (2.19 mL, 23.1 mmol) in THF (100 mL) was refluxed for 8 h. After cooling down to room temperature, 6N HCl (6.6 mL) was added to the reaction mixture and refluxed again for 2 h. After cooling down to room temperature, the reaction mixture was basified with saturated aqueous NaHCO₃ solution (100 mL), extracted with ethyl acetate (400 mL). The extract was washed with brine, dried (Na₂SO₄), filtered, and concentrated to give 1.63 g of colorless amorphous solid. Overall yield of three steps was 92%.

¹H NMR was identical with that of the final compound of Example 1.

This solid (100 mg) was converted to citric acid salt similar to that described in Example 1 to afford 138.9 mg of the citric acid salt as white amorphous solid.

MS (ESI positive) m/z: 333 (M+H)⁺.

IR(KBr): 1732, 1578, 1437, 1394, 1252, 1103, 1072, 756 cm⁻¹

Anal. Calcd for C₂₃H₂₈N₂—C₆H₈O₇.1.5CH₃OH-0.1CH₂Cl₂: C, 63.24; H, 7.32; N, 4.82. Found: C, 63.43; H, 7.49; N, 4.42.

### Example 5

### (3R)-3-(2,3-Dihydro-1′H-spiro[indene-1,4′-piperidin]-1′-ylmethyl)-1,2,3,4-tetrahydroisoquinolin-7-ol

This was prepared according to the procedure described in Example 4 using (3R)-2-(tert-butoxycarbonyl)-7-hydroxy-1,2,3,4-tetrahydroisoquinoline-3-carboxylic acid instead of (3S)-2-(tert-butoxycarbonyl)-1,2,3,4-tetrahydroisoquinoline-3-carboxylic acid. Overall yield was 65.7%.

¹H NMR (270 MHz, DMSO-d6) δ 9.03 (1H, br.s), 7.30-7.10 (4H, m), 6.87 (1H, d, J=8.3 Hz), 6.52 (1H, dd, J=2.1, 8.3 Hz), 6.42 (1H, d, J=2.1 Hz), 3.84 (2H, s), 2.98-2.74 (4H, m), 2.63-2.03 (12H, m), 1.50-1.40 (2H, m).

MS (ESI positive) m/z: 349 (M+H)⁺.

Anal. Calcd for C₂₃H₂₈N₂O-0.2H₂O: C, 78.46; H, 8.13; N, 7.96. Found: C, 78.40; H, 8.12;N, 7.84.

### Example 6

### 3-(2,3-Dihydro-1′H-spiro[indene-1,4′-piperidin]-1′-ylmethyl)-1,2,3,4-tetrahydroisoquinolin-6-ol

### (A) tert-Butyl 3-(2,3-dihydro-1′H-spiro[indene-1,4′-piperidin]-1′-ylcarbonyl)-6-hydroxy-3,4-dihydroisoquinoline-2(1H)-carboxylate

A mixture of 6-hydroxy-1,2,3,4-tetrahydroisoquinoline-3-carboxylic acid (500 mg, 2.59 mmol, this was prepared according to the reported method of J. Org. Chem., 1991, 56, 4388), di-tert-butyl-dicarbonate (622 mg, 2.85 mmol), and Na₂CO₃ (1.4 g, 13 mmol) in dioxane (10 mL) and water (20 mL) was stirred at room temperature for 3 days. The reaction mixture was acidified with 2N HCl (pH 3) and extracted with ethyl acetate (150 mL). The extracts combined were washed with water (50 mL) and brine (50 mL), dried (Na₂SO₄), filtered, and concentrated to give 759.6 mg of the Boc compound as colorless amorphous solid. A mixture of this Boc compound (144 mg, 0.492 mmol), 2,3-dihydrospiro[1H-indene-1,4′-piperidine] hydrochloride (100 mg, 0.447 mmol), WSC (94 mg, 0.492 mmol), triethylamine (0.07 mL, 0.492 mmol), and hydroxybenzotriazole (66 mg, 0.492 mmol) in DMF (5 mL) was stirred at room temperature for 4 days. The reaction mixture was diluted with ethyl acetate (100 mL), washed with water (30 mL), dried (Na₂SO₄), filtered, and concentrated to give 340 mg of yellow gum. This was purified by silica gel column chromatography (n-hexane/ethyl acetate: 2/1) to give 159.2 mg (77%) of title compound as white amorphous solid.

¹H NMR (270 MHz, CDCl₃) δ 9.23 (1H, br.s), 7.80-7.67 (1.5H, m), 7.47-7.36 (1.5H, m), 7.25-7.10 (2.5H, m), 6.86 (0.5H, d, J=7.9 Hz), 6.70-6.53 (1H, m), 5.40-5.20, 5.05-4.25, and 4.18-3.93 (total 5H, each m), 3.45-2.70 (6H, m), 2.15-1.40 (15H, m, including 9H, s at 1.47 ppm).

MS(EI) m/z: 462(M)⁺.

### (B) 3-(2,3-Dihydro-1′H-spiro[indene-1,4′-piperidin]-1′-ylmethyl)-1,2,3,4-tetrahydroisoquinolin-6-ol

This was prepared according to the procedure described in Example 4 using tert-butyl 3-(2,3-dihydro-1′H-spiro[indene-1,4′-piperidin]-11′-ylcarbonyl)-6-hydroxy-3,4-dihydroisoquinoline-2(1H)-carboxylate instead of tert-butyl (3S)-3-(2,3-dihydro-1′H-spiro[indene-1,4′-piperidin]-1′-ylcarbonyl)-3,4-dihydroisoquinoline-2(1H)-carboxylate. Overall yield was 63.3%.

¹H NMR (270 MHz, DMSO-d6) δ 9.05 (1H, br.s), 7.30-7.10 (4H, m), 6.83 (1H, d, J=8.3 Hz), 6.55-6.46 (2H, m), 3.84 (2H, s), 3.04-2.74 (4H, m), 2.65-1.78 (12H, m), 1.50-1.40 (2H, m).

This solid (53.5 mg) was converted to citric acid salt similar to that described in Example 1 to afford 74.6 mg of citric acid salt as white amorphous solid.

MS(EI) m/z: 348(M)⁺.

IR(KBr): 1720, 1578, 1508, 1477, 1456, 1389, 1306, 1242 cm⁻¹

Anal. Calcd for C₂₃H₂₈N₂O—C₆H₈O₇-2H₂O: C, 60.40; H, 6.99; N, 4.86. Found: C, 60.15; H, 6.86; N, 4.57.

### Example 7

### 5-Bromo-3-(2,3-dihydro-1′H-spiro[indene-1,4′-piperidin]-1′-ylmethyl)-1,2,3,4-tetrahydroisoquinolin-8-ol

This was prepared according to the procedure described in Example 6 using 5-bromo-8-hydroxy-1,2,3,4-tetrahydroisoquinoline-3-carboxylic acid (this was prepared according to the reported method of Tetrahedron Lett., 2001, 42, 5797) instead of 6-hydroxy-1,2,3,4-tetrahydroisoquinoline-3-carboxylic acid. Overall yield was 10.8%.

¹H NMR (270 MHz, DMSO-d6) δ 9.60 (1H, br.s), 7.28-7.09 (5H, m), 6.60 (1H, d, J=8.6 Hz), 3.96 (1H, d, J=16.3 Hz), 3.63 (1H, d, J=16.5 Hz), 2.98-2.75 (4H, m), 2.70-1.78 (12H, m), 1.50-1.40 (2H, m).

This solid (50.3 mg) was converted to citric acid salt similar to that described in Example 1 to afford 59.4 mg of citric acid salt as white solid.

MS (ESI positive) m/z: 427, 429 (M+H)⁺.

IR(KBr): 1719, 1585, 1477, 1448, 1296, 1186 cm⁻¹

Anal. Calcd for C₂₃H₂₇BrN₂O—C₆H₈O₇-2H₂O: C, 53.13; H, 6.00; N, 4.27. Found: C, 53.39; H, 5.89; N, 4.17.

### Example 8

### 3-(2,3-Dihydro-1′H-spiro[indene-1,4′-piperidin]-1′-ylmethyl)-2-methyl-1,2,3,4-tetrahydroisoquinolin-8-ol

A mixture of 5-bromo-3-(2,3-dihydro-1′H-spiro[indene-1,4′-piperidin]-1′-ylmethyl)-1,2,3,4-tetrahydroisoquinolin-8-ol (140 mg, 0.328 mmol), di-tert-butyl-dicarbonate (179 mg, 0.82 mmol), and 2N NaOH (5 mL) in dioxane (5 mL) was stirred at room temperature for 4 days. The reaction mixture was extracted with ethyl acetate (100 mL). The extracts combined were washed with water (20 mL) and brine (20 mL), dried (Na₂SO₄), filtered, and concentrated to give 145.6 mg (84.2%) of the Boc compound as pale yellow amorphous solid. A suspension mixture of this Boc compound (145 mg, 0.275 mmol) and 5% Pd/C (15 mg) in methanol (20 mL) was stirred under hydrogen atmosphere at room temperature for 16 h. After removal of the catalyst using Celite filtration, the filtrate was concentrated. The residue was purified by silica gel column chromatography (CH₂Cl₂/methanol/NH₄OH: 400/10/1) to give 47.1 mg (47.2%) of title compound as white amorphous solid and 64.3 mg (52.3%) of the N-Boc derivative.

¹H NMR (270 MHz, CDCl₃) δ 7.24-7.11 (4H, m), 6.99 (1H, dd, J=7.7, 7.8 Hz), 6.69 (1H, d, J=7.4 Hz), 6.54 (1H, d, J=7.8 Hz), 3.85 (1H, d, J=16.4 Hz), 3.69 (1H, d, J=16.4 Hz), 3.08-2.60 (8H, m, including 2H, t, J=7.2 Hz at 2.88 ppm), 2.51 (3H, s), 2.38-1.85 (7H, m, including 2H, t, J=7.6 Hz at 1.99 ppm), 1.57-1.45 (2H, m).

This solid (45 mg) was converted to citric acid salt similar to that described in Example 1 to afford 51 mg of citric acid salt as white solid.

MS (ESI positive) m/z: 363 (M+H)⁺.

IR(KBr): 1718, 1597, 1472, 1439, 1389, 1346, 1285, 1207 cm⁻¹

Anal. Calcd for C₂₄H₃₀N₂O—C₆H₈O₇-2.1H₂O: C, 60.82; H, 7.18; N, 4.73. Found C, 60.51; H, 7.03; N, 4.46.

### Example 9

### 3-(2,3-Dihydro-1′H-spiro[indene-1,4′-piperidin]-1′-ylmethyl)-1,2,3,4-tetrahydroisoquinolin-8-ol

A mixture of tert-butyl 3-(2,3-dihydro-1′H-spiro[indene-1,4′-piperidin]-1′-ylmethyl)-8-hydroxy-3,4-dihydroisoquinoline-2(1H)-carboxylate (64 mg, 0.143 mmol, this was prepared in Example 8 as the N-Boc derivative) and 10% HCl solution in methanol (3 mL) was stirred at room temperature for 18 h. After evaporation of the solvent, the residue was basified with 2N NaOH and extracted with ethyl acetate (100 mL). The extracts combined were washed with brine, dried (Na₂SO₄), filtered, and concentrated to give 49.8 mg (100%) of title product as brown amorphous solid.

¹H NMR (270 MHz, CDCl₃)δ 7.28-7.11 (4H, m), 6.92 (1H, dd, J=7.7, 7.8 Hz), 6.61 (1H, d, J=7.4 Hz), 6.55 (1H, d, J=7.9 Hz), 4.23 (1H, d, J=15.8 Hz), 3.89 (1H, d, J=16.0 Hz), 3.20-3.05 (1H, m), 3.00-2.30 (10H, m, including 2H, t, J=7.2 Hz at 2.89 ppm), 2.20-1.90 (5H, m, including 2H, t, J=7.3 Hz at 2.00 ppm), 1.60-1.47 (2H, m).

This solid (49 mg) was converted to citric acid salt similar to that described in Example 1 to afford 52 mg of citric acid salt as pale yellow amorphous solid.

MS (ESI positive) m/z: 349 (M+H)⁺.

IR(KBr): 3200, 1719, 1597, 1472, 1439, 1375, 1342, 1283 cm⁻¹

Anal. Calcd for C₂₃H₂₈N₂O—C₆H₈O₇-2H₂O: C, 60.40; H, 6.99; N, 4.86. Found: C, 60.32; H, 6.88; N, 4.59.

### Example 10

### 3-(2,3-Dihydro-1′H-spiro[indene-1,4′-piperidin]-1′-ylmethyl)-5-fluoro-1,2,3,4-tetrahydroisoquinolin-8-ol

### (A) Diethyl (2-fluoro-5-methoxybenzyl)(formylamino)malonate

To a stirred solution of 2-fluoro-5-methoxybenzyl alcohol (3.00 g, 19.2 mmol) and triethylamine (3.75 mL, 26.9 mmol) in CH₂Cl₂ (30 mL) was added methanesulfonyl chloride (1.78 mL, 23 mmol) at 0° C. and the stirring was continued for 1 h at 0° C. The reaction mixture was quenched with water and extracted with CH₂Cl₂. The extracts combined were dried (Na₂SO₄), filtered, and concentrated to give 4.48 g of mesylate as a colorless oil. To a stirred solution of diethyl formamidomalonate (4.68 g, 23 mmol) in DMF (20 mL) was added NaH (60% oil suspension, 0.85 g, 21 mmol) at 0° C. After 10 min stirring, the solution of mesylate prepared above (4.48 g) in DMF (10 mL) was added to the reaction mixture 0° C. The reaction mixture was stirred at 0° C. for 30 min and then at room temperature for 16 h. The reaction mixture was quenched with water and extracted with ethyl acetate. The extracts combined were washed with water and brine, dried (MgSO₄), filtered, and concentrated to give white solid. This was purified by silica gel column chromatography (n-hexane/ethyl acetate:3/1) to afford 5.07 g (78%) of title compound as a white solid.

¹H NMR (270 MHz, CDCl₃) δ 8.16 (1H, s), 6.90 (1H, dd, J=9.1, 9.2 Hz), 6.77-6.68 (2H, m), 6.62-6.56 (1H, m), 4.36-4.22 (4H, m), 3.73 (3H, s), 3.69 (2H, s), 1.30 (6H, t, J=7.1 Hz).

### (B) 2-(tert-Butoxycarbonyl)-5-fluoro-8-hydroxy-1,2,3,4-tetrahydroisoquinoline-3-carboxylic Acid

A mixture of diethyl (2-fluoro-5-methoxybenzyl)(formylamino)malonate (5.07 g, 14.9 mmol), c-HCl (40 mL), and acetic acid (20 mL) was stirred at 100° C. for 20 h and subsequently at 120° C. for 4 h. After cooling down to room temperature, the reaction mixture was diluted with water and washed with CH₂Cl₂, and concentrated to give 3.70 g of mixture of 2-fluoro-5-methoxyphenylalanine hydrochloride and 2-fluoro-5-hydroxyphenylalanine hydrochloride. A mixture of the above mixture (3.70 g), 37% formalin (17 mL), and water (17 mL) was heated to 90° C. After 3 h heating, 0.5 mL of 2N HCl was added to the reaction mixture. After 1 h stirring at 90° C., the reaction mixture was cooled, concentrated, and dried in vacuo to give a mixture of 5-fluoro-8-methoxy-1,2,3,4-tetrahydroisoquinoline-3-carboxylic acid and 5-fluoro-8-hydroxy-1,2,3,4-tetrahydroisoquinoline-3-carboxylic acid as yellow solid. To a stirred suspension of this mixture in methanol (50 mL) was added dropwise thionyl chloride (5.4 mL, 74.5 mmol) at room temperature. After 2.5 h stirring with reflux, the reaction mixture was concentrated. The residue was basified with aqueous NaHCO₃ solution and extracted with ethyl acetate. The extracts combined were dried (MgSO₄), filtered, and concentrated to give 3.08 g of the methyl esters as an orange color oil. To a stirred solution of the methyl esters (3.08 g) and triethylamine (5.4 mL, 39 mmol) in CH₂Cl₂ (30 mL) was added di-tert-butyl-dicarbonate (3.50 g, 16 mmol) at room temperature. After 16 h stirring, the reaction mixture was quenched with aqueous NaHCO₃ solution and extracted with CH₂Cl₂. The extracts combined were dried (MgSO₄), filtered, and concentrated. The residue was purified by silica gel column chromatography (n-hexane/ethyl acetate:6/1) to afford 3.02 g (60%) of the Boc esters as a white solid. A mixture of the Boc esters (3.02 g), 2N NaOH (18 mL), THF (20 mL), and methanol (20 mL) was stirred at room temperature for 1 h. After evaporation of the solvent, the residue was dissolved in water, acidified with 10% aqueous solution of citric acid, and extracted with CH₂Cl₂. The extracts combined were dried (MgSO₄), filtered, and concentrated to give 2.60 g (90%) of the carboxylic acids as a white solid. This carboxylic acids mixture was separated by silica gel column chromatography (CH₂CH₂Cl₂/methanol: 40/1) to give 1.46 g (30% for 5 steps) of 2-(tert-butoxycarbonyl)-5-fluoro-8-methoxy-1,2,3,4-tetrahydroisoquinoline-3-carboxylic acid and 720 mg (16% for 5 steps) of the title compound.

Title Compound:

¹H NMR (300 MHz, CDCl₃) δ 6.78-6.66 (1H, m), 6.60-6.50 (1H, m), 5.25-5.10 (0.5H, m), 5.05-4.95 (0.5H, m), 4.75-4.57 (1H, m), 4.45-4.20 (1H, m), 3.50-3.30 (1H, m), 3.00-2.80 (1H, m), 1.52 and 1.46 (total 9H, each s).

MS (ESI negative) m/z: 310 (M−H)⁻.

Methoxy Derivative:

¹H NMR (300 MHz, CDCl₃) δ 6.92-6.81 (1H, m), 6.67-6.59 (1H, m), 5.30-5.22 (0.7H, m), 5.07-4.98 (0.3H, m), 4.78-4.57 (1H, m), 4.38-4.25 (1H, m), 3.80 and 3.78 (total 3H, each br.s), 3.54-3.35 (1H, m), 3.03-2.84 1H, m), 1.52 and 1.47 (total 9H, each s).

MS (ESI negative) m/z: 324 (M−H)⁻.

### (C) 3-(2,3-Dihydro-1′H-spiro[indene-1,4′-piperidin]-1′-ylmethyl)-5-fluoro-1,2,3,4-tetrahydroisoquinolin-8-ol

This was prepared according to the procedure described in Example 4 using 2-(tert-butoxycarbonyl)-5-fluoro-8-hydroxy-1,2,3,4-tetrahydroisoquinoline-3-carboxylic acid instead of 2-(tert-butoxycarbonyl)-1,2,3,4-tetrahydroisoquinoline-3-carboxylic acid and trifluoroacetic acid instead of HCl solution in methanol for the removal of the Boc group. Overall yield was 62.1%.

¹H NMR (270 MHz, DMSO-d6) δ 9.25 (1H, br.s), 7.26-7.08 (4H, m), 6.77 (1H, dd, J=9.0, 9.1 Hz), 6.56 (1H, dd, J=4.8, 8.9 Hz), 3.94 (1H, d, J=16.5 Hz), 3.62 (1H, d, J=16.5 Hz), 3.00-2.62 (6H, m, including 2H, t, J=7.3 Hz at 2.84 ppm), 2.42-2.37 (2H, m), 2.30-1.78 (7H, m), 1.50-1.40 (2H, m).

This solid (73.3 mg) was converted to citric acid salt similar to that described in Example 1 to afford 100 mg of citric acid salt as white solid.

MS (ESI positive) m/z: 367 (M+H)⁺.

IR(KBr): 3423, 1719, 1578, 1477, 1438, 1375, 1246, 760 cm⁻¹

Anal. Calcd for C₂₃H₂₇FN₂O—C₆H₈O₇—H₂O: C, 60.41; H, 6.47; N, 4.86. Found: C, 60.16; H, 6.40; N, 4.63.

### Example 11

### 5-Fluoro-3-[(1-methyl-1,2-dihydro-1′H-spiro[indole-3,4′-piperidin]-1′-yl)methyl]-1,2,3,4-tetrahydroisoquinolin-8-ol

This was prepared according to the procedure described in Example 10 using 1-methyl-1,2-dihydrospiro[indole-3,4′-piperidine] instead of 2,3-dihydrospiro[indene-1,4′-piperidine]. Overall yield was 46%.

¹H NMR (270 MHz, DMSO-d6) δ 9.25 (1H, br.s), 7.07-6.97 (2H, m), 6.76 (1H, dd, J=8.7, 9.1 Hz), 6.65-6.52 (2H, m), 6.48 (1H, d, J=7.7 Hz), 3.94 (1H, d, J=16.6 Hz), 3.62 (1H, d, J=16.3 Hz), 3.17 (2H, s), 2.95-2.60 (7H, m, including 3H, s, at 2.70 ppm), 2.46-2.34 (2H, m), 2.24-1.99 (3H, m), 1.90-1.76 (2H, m), 1.65-1.53 (2H, m).

This solid (104 mg) was converted to citric acid salt similar to that described in Example 1 to afford 150 mg of citric acid salt as white solid.

MS (ESI positive) m/z: 382 (M+H)⁺.

IR(KBr): 3489, 1719, 1578, 1475, 1364, 1244, 739 cm⁻¹

Anal. Calcd for C₂₃H₂₈FN₃O—C₆H₈O₇-2.5H₂O: C, 56.30; H, 6.68; N, 6.79. Found: C, 56.17; H, 6.61; N, 6.71.

### Example 12

### 5-Chloro-3-(2,3-dihydro-1′H-spiro[indene-1,4′-piperidin]-1′-ylmethyl)-1,2,3,4-tetrahydroisoquinolin-8-ol

### (A) Diethyl (acetylamino)(5-{[tert-butyl(dimethyl)silyl]oxy}-2-chlorobenzyl)malonate

This was prepared according to the procedure described in Example 10 using [3-(bromomethyl)-4-chlorophenoxy](tert-butyl)dimethylsilane (this was prepared according to the reported method of J. Org. Chem., 1996, 61, 6974) instead of 2-fluoro-5-methoxybenzyl methanesulfonate and diethyl acetamidomalonate instead of diethyl formamidomalonate. Yield was 59%.

¹H NMR (300 MHz, CDCl₃) δ 7.16 (1H, d, J=8.6 Hz), 6.66 (1H, dd, J=2.8, 8.6 Hz), 6.57 (1H, d, J=2.8 Hz), 6.54 (1H, br.s), 4.37-4.07 (4H, m), 3.78 and 3.74 (total 2H, each s), 2.05 and 2.01 (total 3H, each s), 1.29 (6H, t, J=8.8 Hz), 0.96 (9H, s), 0.17 (6H, s).

### (B) 2-(tert-Butoxycarbonyl)-5-chloro-8-hydroxy-1,2,3,4-tetrahydroisoquinoline-3-carboxylic acid and 2-(tert-butoxycarbonyl)-8-[(tert-butoxycarbonyl)oxy]-5-chloro-1,2,3,4-tetrahydroisoquinoline-3-carboxylic acid

This was prepared according to the procedure described in Example 10 using diethyl (acetylamino)(5-{[tert-butyl(dimethyl)silyl]oxy}-2-chlorobenzyl)malonate (this was prepared in the former reaction step) instead of diethyl (2-fluoro-5-methoxybenzyl)(formylamino)malonate and without esterification (thionyl chloride, methanol). Overall yield of the N-Boc derivative and the N,O-di-Boc derivative was 94.9%. The N-Boc derivative was major product.

¹H NMR (300 MHz, DMSO-d6) δ 10.00 (1H, s), 7.44 and 7.13 (total 1H, each d, J=8.7 and 8,6 Hz), 7.18 and 6.72 (total 1H, each d, J=8.8 Hz), 5.08-5.00 (0.5H, m), 4.90-4.80 (0.5H, m), 4.50-4.35 (1H, m), 4.30-4.15 (1H, m), 3.50-2.80 (2H, m), 1.50, 1.46 and 1.41 (total 9H, each s).

MS(ESI negative) m/z: 326, 426 (M−H)⁻.

### Example 12

### 5-Chloro-3-(2,3-dihydro-1′H-spiro[indene-1,4′-piperidin]-1′-ylmethyl)-1,2,3,4-tetrahydroisoquinolin-8-ol

This was prepared according to the procedure described in Example 4 using 2-(tert-butoxycarbonyl)-5-chloro-8-hydroxy-1,2,3,4-tetrahydroisoquinoline-3-carboxylic acid and 2-(tert-butoxycarbonyl)-8-[(tert-butoxycarbonyl)oxy]-5-chloro-1,2,3,4-tetrahydroisoquinoline-3-carboxylic acid instead of (3S)-2-(tert-butoxycarbonyl)-1,2,3,4-tetrahydroisoquinoline-3-carboxylic acid. Overall yield was 51.1%.

¹H NMR (300 MHz, DMSO-d6) δ 9.60 (1H, br.s), 7.27-7.10 (4H, m), 7.06 (1H, d, J=8.4 Hz), 6.64 (1H, d, J=9.0 Hz), 3.96 (1H, d, J=15.6 Hz), 3.63 (1H, d, J=16.8 Hz), 3.00-2.65 (7H, m, including 2H, t, J=7.3 Hz at 2.85 ppm), 2.45-2.37 (2H, m), 2.30-2.05 (3H, m), 1.97 (2H, t, J=7.2 Hz), 1.93-1.80 (2H, m), 1.50-1.40 (2H, m).

This solid (86.8 mg) was converted to citric acid salt similar to that described in Example 1 to afford 119.8 mg of citric acid salt as white amorphous solid.

MS (ESI positive) m/z: 383 (M+H)⁺.

IR(KBr): 2945, 1719, 1600, 1456, 1340, 1298, 1192, 760 cm⁻¹

### Example 13

### 5-Chloro-3-(1′H,3H-spiro[2-benzofuran-1,4′-piperidin]-1′-ylmethyl)-1,2,3,4-tetrahydroisoquinolin-8-ol

This was prepared according to the procedure described in Example 12 using 3H-spiro[2-benzofuran-1,4′-piperidine] instead of 2,3-dihydrospiro[indene-1,4′-piperidine]. Overall yield was 22.9%.

¹H NMR (270 MHz, DMSO-d6) δ 9.58 (1H, br.s), 7.28 (4H, s), 7.05 (1H, d, J=8.6 Hz), 6.63 (1H, d, J=8.6 Hz), 4.97 (2H, s), 3.95 (1H, d, J=16.5 Hz), 3.62 (1H, d, J=15.2 Hz), 2.98-2.63 (5H, m), 2.50-1.85 (7H, m), 1.68-1.58 (2H, m).

This solid (76 mg) was converted to citric acid salt similar to that described in Example 1 to afford 100.8 mg of citric acid salt as white amorphous solid.

MS (ESI positive) m/z: 385 (M+H)⁺.

IR(KBr): 3410, 1720, 1591, 1578, 1456, 1369, 1298 cm⁻¹

Anal. Calcd for C₂₂H₂₅ClN₂O₂—C₆H₈O₇-1.8H₂O: C, 55.18; H, 6.05; N, 4.60. Found: C, 54.92; H, 6.06; N, 4.45.

### Example 14

### 5-Chloro-3-[(1-methyl-1,2-dihydro-1′H-spiro[indole-3,4′-piperidin]-1′-yl)methyl]-1,2,3,4-tetrahydroisoquinolin-8-ol

This was prepared according to the procedure described in Example 12 using 1-methyl-1,2-dihydrospiro[indole-3,4′-piperidine] instead of 3H-spiro[2-benzofuran-1,4′-piperidine]. Overall yield was 38.9%.

¹H NMR (300 MHz, DMSO-d6) δ 7.10-6.96 (3H, m), 6.54-6.51 (2H, m), 6.48 (1H, d, J=8.1 Hz), 3.95 (1H, d, J=16.3 Hz), 3.61 (1H, d, J=16.5 Hz), 3.17 (2H, s), 2.97-2.62 (7H, m, including 3H, s, at 2.71 ppm), 2.40 (2H, d, J=6.2 Hz), 2.25-1.98 (4H, m), 1.90-1.77 (2H, m), 1.65-1.55 (2H, m).

This solid (80 mg) was converted to citric acid salt similar to that described in Example 1 to afford 101.7 mg of citric acid salt as white amorphous solid.

MS (ESI positive) m/z: 412 (M+H)⁺.

IR(KBr): 3392, 1720, 1591, 1578, 1454, 1381, 1283 cm⁻¹

Anal. Calcd for C₂₃H₂₈ClN₃O—C₆H₈O₇-3.5H₂O-0.2CH₂Cl₂: C, 52.34; H, 6.53; N, 6.27. Found: C, 52.08; H, 6.14; N, 5.97.

### Example 15

### 1′-{[1-Methyl-1,2,3,4-tetrahydroisoquinolin-3-yl]methyl}-2,3-dihydrospiro[indene-1,4′-piperidine]

To the stirred solution of 1′-(1,2,3,4-tetrahydroisoquinolin-3-ylmethyl)-2,3-dihydrospiro[indene-1,4′-piperidine] (1.11 g, 3.34 mmol, this was prepared as Example 1) in 2N NaOH solution (5 mL) and dioxane (10 mL) was added di-tert-butyl-dicarbonate (800 mg, 3.67 mmol) at room temperature. After 18 h stirring, dioxane was removed by evaporation and the residue was extracted with ethyl acetate. The extracts combined were washed with brine, dried (Na₂SO₄), filtered, and concentrated to give 1.40 g of colorless oil. This was purified by silica gel column chromatography (n-hexane/ethyl acetate:4/1) to afford 1.00 g (69.4%) of the Boc derivative as a white amorphous solid. To the stirred solution of this Boc derivative (100 mg, 0.23 mmol) in THF (5 mL) was added 1.47 M solution of tert-butyllithium (0.19 mL, 0.28 mmol) at −78° C. After 1 h stirring at −78° C., iodomethane (0.07 mL, 0.46 mmol) was added to the reaction mixture and gradually warmed to room temperature. After 18 h stirring, the reaction mixture was quenched with water (5 mL) and extracted with ethyl acetate. The extracts combined were washed with brine, dried (Na₂SO₄), filtered, and concentrated to give 115 mg of yellow oil. This was purified by silica gel column chromatography (n-hexane/ethyl acetate:20/1) followed by preparative TLC (n-hexane/ethyl acetate:4/1) to afford 44.9 mg (43.7%) of the 1-methyl derivative as a colorless oil. The mixture of this 1-methyl derivative (44 mg, 0.099 mmol) and 10% HCl solution in methanol (3 mL) was stirred at room temperature for 18 h. The reaction mixture was concentrated, and the residue was basified with 2N NaOH and extracted with ethyl acetate. The extracts combined were washed with brine, dried (Na₂SO₄), filtered, and concentrated to give 30.7 mg (89.5%)of pale brown oil.

¹H NMR (300 MHz, CDCl₃) δ 7.30-7.05 (8H, m), 4.32 (0.65H, q, J=6.8 Hz), 4.16 (0.35H, q, J=6.4 Hz), 3.43-3.32 (0.65H, m), 3.20-3.08 (0.35H, m), 3.00-2.30 (8H, m), 2.22-1.86 (7H, m), 1.60-1.45 (5H, m, including 1.05H, d, J=6.4 Hz at 1.52 ppm and 1.95H, d, J=6.8 Hz at 1.47 ppm).

This solid (30 mg) was converted to citric acid salt similar to that described in Example 1 to afford 42 mg of citric acid salt as white amorphous solid.

MS (ESI positive) m/z: 347 (M+H)⁺.

IR(KBr): 2934, 1719, 1578, 1364, 1209, 1096, 762 cm⁻¹

### Example 16

### 3-[(3,3-Dimethyl-1′H,3H-spiro[2-benzofuran-1,4′-piperidin]-1′-yl)methyl]-1,2,3,4-tetrahydroisoquinolin-8-ol

### (A) 5-Bromo-3-[(3,3-dimethyl-1′H,3H-spiro[2-benzofuran-1,4′-piperidin]-1′-yl)methyl]-1,2,3,4-tetrahydroisoquinolin-8-ol

This was prepared according to the procedure described in Examples 7 and 10 using 3,3-dimethyl-3H-spiro[2-benzofuran-1,4′-piperidine] (this was prepared by de-methylation of 1′,3,3-trimethyl-3H-spiro[2-benzofuran-1,4′-piperidine] using the following reaction condition: 1-chloroethyl chloroformate in 1,2-dichloroethane followed by methanol reflux) instead of 2,3-dihydrospiro[indene-1,4′-piperidine]. Overall yield was 47.3%.

¹H NMR (300 MHz, CDCl₃) δ 7.30-7.08 (5H, m), 6.46 (1H, d, J=8.3 Hz), 4.30-3.50 (3H, m), 3.20-2.30 (9H, m), 2.20-1.95 (2H, m), 1.76-1.64 (2H, m), 1.51 (6H,s).

MS (ESI positive) m/z: 457,459 (M+H)⁺.

### (B) 3-[(3,3-Dimethyl-1′H,3H-spiro[2-benzofuran-1,4′-piperidin]-1′-yl)methyl]-1,2,3,4-tetrahydroisoquinolin-8-ol

A mixture of 5-bromo-3-[(3,3-dimethyl-1′H,3H-spiro[2-benzofuran-1,4′-piperidin]-1′-yl)methyl]-1,2,3,4-tetrahydroisoquinolin-8-ol (this was prepared in preparation 7, 98.9 mg, 0.216 mmol) and 10% palladium on carbon (9.9 mg) in ethyl acetate (5 mL) was stirred under hydrogen atmosphere at room temperature. After 1 d stirring, 10% palladium on carbon (10 mg) was added to the reaction mixture. After 6 h stirring, 10% palladium on carbon (10 mg) was added to the reaction mixture. After Celite filtration, the filtrate was concentrated. A mixture of this residue and 10% palladium on carbon (20 mg) in ethyl acetate (6 mL) was stirred under hydrogen atmosphere at room temperature for 7 h. After Celite filtration, the filtrate was concentrated to give 87.3 mg of crude product, which was solidified by adding ethyl acetate. This solid was collected by filtration and dried to give 72.5 mg (89%) of title compound as white.

¹H NMR (270 MHz, DMSO-d6) δ 9.92 (1H, br.s), 7.34-7.14 (4H, m), 7.09 (1H, dd, J=7.7, 7.9 Hz), 6.73 (1H, d, J=7.7 Hz), 6.68 (1H, d, J=7.4 Hz), 4.16 (1H, d, J=16.5 Hz), 4.07 (1H, d, J=16.3 Hz), 3.69 (1H, br.s), 3.03-2.90 (2H, m), 2.82-2.36 (7H, m), 2.12-1.98 (2H, m), 1.65-1.50 (2H, m), 1.45 (6H, s).

This solid was converted to citric acid salt similar to that described in Example 1 to afford citric acid salt as white solid.

MS (ESI positive) m/z: 379 (M+H)⁺.

Anal. Calcd for C₃₀H₃₈N₂O₉—C₆H₈O₇-2.5H₂O-2CH₂Cl₂: C, 48.93; H, 6.03; N, 3.57. Found: C, 48.65; H, 5.77; N, 3.47.

### Example 17

### 3-[(1′-Methyl-1′,2′-dihydro-1H-spiro[piperidine-4,3′-pyrrolo[2,3-b]pyridin]-1-yl)methyl]-1,2,3,4-tetrahydroisoquinolin-8-ol

### (A) 1-Benzyl-1′,2′-dihydrospiro[piperidine-4,3′-pyrrolo[2,3-b]pyridine]

A mixture of (2-chloropyridin-3-yl)acetonitrile (830 mg, 5.44 mmol, reported by Bremner, H. D. et al., Synthesis, 1992, 528), N-benzyl-N,N-bis(2-chloroethyl)amine (1389 mg, 5.98 mmol), and hexadecyltributylphosphonium bromide (138.1 mg, 0.272 mmol) in 50% NaOH solution (8.3 mL) was stirred at 100° C. for 1 h (J. Heterocycle Chem, 1986, 23, 73). The reaction mixture was diluted with water (30 mL) and extracted with ethyl acetate (30 mL×3). The extracts combined were dried (Na₂SO₄), filtered, and concentrated to give 2.293 g of crude product, which was purified by silica gel column chromatography (hexane/ethyl acetate: 1/1) to give 1026.6 mg (60%) of benzyl-4-(2-chloropyridin-3-yl)piperidine-4-carbonitrile as a brown solid.

¹H NMR (270 MHz, CDCl₃) δ 8.40 (1H, dd, J=1.8, 4.6 Hz), 7.75 (1H, dd, J=1.8, 7.9 Hz), 7.36-7.23 (6H, m), 3.62 (2H, s), 3.90-3.00 (2H, m), 2.67-2.46 (4H, m), 2.16-2.04 (2H, m).

To a stirred suspension of LiAlH₄ (478.2 mg, 12.6 mmo) in THF (30 mL) was added a solution of benzyl-4-(2-chloropyridin-3-yl)piperidine-4-carbonitrile (983.6 mg, 3.15 mmol) in THF (20 mL) at 0° C. and the reaction mixture was refluxed for 15 h. After cooled down to 0° C., the reaction mixture was quenched with aqueous Na₂SO₄ solution (3.6 mL). The reaction mixture was diluted with CH₂Cl₂ (20 mL) and dried (Na₂SO₄). After Celite filtration, the filtrate was concentrated to give 1.0677 g of the amine derivative, which was dissolved in acetonitrile (20 mL) and refluxed in the presence of K₂CO₃ (1.306 g, 9.45 mmol) for 21 h. After Celite filtration, the filtrate was concentrated to give 1.0483 g of crude product as the title compound.

¹H NMR (270 MHz, CDCl₃) δ 7.82 (1H, dd, J=1.5, 5.3 Hz), 7.35-7.20 (6H, m), 6.53 (1H, dd, J=4.8, 6.4 Hz), 4.45 (2H, br.s), 3.55 (2H, s), 3.45 (2H, s), 3.90-3.00 (2H, m), 2.88-2.79 (2H, m), 2.18-2.07 (2H, m), 1.96-1.84 (2H, m), 1.80-1.65 (2H, m).

MS (ESI positive) m/z: 280 (M+H)⁺.

### (B) 1′-Methyl-1′,2′-dihydrospiro[piperidine-4,3′-pyrrolo[2,3-b]pyridine]

To a stirred suspension of NaH (60% oil suspension, 7.3 mg, 0.183 mmol) in DMF (0.5 mL) was added a solution of 1-benzyl-1′,2′-dihydrospiro[piperidine-4,3′-pyrrolo[2,3-b]pyridine] (this was prepared in the former reaction step, 46.4 mg, 0.166 mmol) in DMF (2.5 mL) at 0° C. and the mixture was stirred at room temperature for 0.5 h. To this reaction mixture was added a solution of iodomethane (26 mg, 0.183 mmol) in DMF (2.0 mL) at 0° C., and the reaction mixture was stirred at room temperature for 21 h. The reaction mixture was quenched with aqueous NaHCO₃ solution (20 mL), extracted with CH₂Cl₂ (15 mL×3), dried (Na₂SO₄), filtered, and concentrated. The residue dissolved in ethyl acetate (30 mL) was washed with water (30 mL) and brine (30 mL), dried (Na₂SO₄), filtered, and concentrated to give 54.5 mg of crude product, which was purified by preparative TLC (CH₂Cl₂/methanol: 15/1) to afford 10.6 mg (22%) of 1-benzyl-1′-methyl-1′,2′-dihydrospiro[piperidine-4,3′-pyrrolo[2,3-b]pyridine] as colorless oil.

¹H NMR (270 MHz, CDCl₃) δ 7.88 (1H, dd, J=1.5, 5.3 Hz), 7.37-7.24 (5H, m), 7.15 (1H, dd, J=1.6, 7.1 Hz), 6.44 (1H, dd, J=5.3, 7.1 Hz), 3.55 (2H, s), 3.30 (2H, s), 2.95 (3H, s), 2.89-2.79 (2H, m), 2.22-2.08 (2H, m), 1.97-1.64 (4H, m).

A mixture of 1-benzyl-1′-methyl-1′,2′-dihydrospiro[piperidine-4,3′-pyrrolo[2,3-b]pyridine] (237.1 mg, 0.808 mmol) and 1-chloroethyl chloroformate (5 mL) was stirred at 100° C. for 21 h. After evaporation of the 1-chloroethyl chloroformate, the residue was dissolved in methanol (15 mL) and refluxed for 1 d. After evaporation of the solvent, the residue was basified with aqueous NaHCO₃ solution and extracted with CH₂Cl₂ (15 mL×3). The extracts combined were dried (Na₂SO₄), filtered, and concentrated to give 69.1 mg of crude product. The aqueous layer was concentrated to give 178.1 mg of crude product. These crude products were purified by preparative TLC (CH₂Cl₂/methanol: 10/1) to afford 105.5 mg (64%) of title compound as yellow oil.

¹H NMR (300 MHz, CDCl₃) δ 7.89 (1H, dd, J=1.5, 5.3 Hz), 7.15 (1H, dd, J=1.7, 7.0 Hz), 6.46 (1H, dd, J=5.3, 7.0 Hz), 3.35 (2H, s), 3.10-3.01 (2H, m), 2.96 (3H, s), 2.83-2.72 (2H, m), 1.90 (1H, br.s), 1.86-1.62 (4H, m).

MS (ESI positive) m/z: 204 (M+H)⁺.

### (C) 5-Bromo-3-[(1′-methyl-1′,2′-dihydro-1H-spiro[piperidine-4,3′-pyrrolo[2,3-b]pyridin]-1-yl)methyl]-1,2,3,4-tetrahydroisoquinolin-8-ol

This was prepared according to the procedure described in Example 16 using 1′-methyl-1′,2′-dihydrospiro[piperidine-4,3′-pyrrolo[2,3-b]pyridine] instead of 3,3-dimethyl-3H-spiro[2-benzofuran-1,4′-piperidine]. 69.3 mg (18.9%) of the title compound was obtained as pale yellow oil.

¹H NMR (300 MHz, CDCl₃) δ 7.87 (1H, dd, J=1.5, 5.3 Hz), 7.25-7.10 (2H, m), 6.55-6.42 (2H, m), 4.30-1.65 (21H, m, including 1H, d, J=18 Hz at 4.15 ppm, 1H, d, J=18 Hz at 3.88 ppm, 2H, s, at 3.32 ppm, 3H, s, at 2.95 ppm).

MS (ESI positive) m/z: 443,445 (M+H)⁺.

### (D) 3-[(1′-Methyl-1′,2′-dihydro-1H-spiro[piperidine-4,3′-pyrrolo[2,3-b]pyridin]-1-yl)methyl]-1,2,3,4-tetrahydroisoquinolin-8-ol

This was prepared according to the procedure described in Examples 8 and 9 using 5-bromo-3-[(1′-methyl-1′,2′-dihydro-1H-spiro[piperidine-4,3′-pyrrolo[2,3-b]pyridin]-1-yl)methyl]-1,2,3,4-tetrahydroisoquinolin-8-ol (this was prepared in the former reaction step) instead of 5-bromo-3-(2,3-dihydro-1′H-spiro[indene-1,4′-piperidin]-1′-ylmethyl)-1,2,3,4-tetrahydroisoquinolin-8-ol. Overall yield was 47.5%.

¹H NMR (270 MHz, CDCl₃) δ 7.89 (1H, dd, J=1.6, 5.4 Hz), 7.17 (1H, dd, J=1.6, 7.3 Hz), 6.97 (1H, dd, J=7.8, 7.8 Hz), 6.66 (1H, d, J=7.6 Hz), 6.56 (1H, d, J=7.9 Hz), 6.46 (1H, dd, J=5.4, 7.1 Hz), 4.22 (1H, d, J=15.8 Hz), 3.89 (1H, d, J=16.3 Hz), 3.32 (2H, s), 3.13-1.65 (17H, m, including 3H, s, at 2.96 ppm).

This solid was converted to citric acid salt similar to that described in Example 1 to afford citric acid salt as white solid.

MS (ESI positive) m/z: 365 (M+H)⁺.

IR(KBr): 3400, 1717, 1663, 1582, 1472, 1352, 1285, 777 cm⁻¹

Anal. Calcd for C₂₂H₂₈N₄O—C₆H₈O₇-2.5H₂O-0.3CH₂Cl₂: C, 54.20; H, 6.69; N, 8.93. Found: C, 54.38; H, 6.64; N, 8.53.

### Example 18

### 3-[(6-Fluoro-1′H,3H-spiro[2-benzofuran-1,4′-piperidin]-1′-yl)methyl]-1,2,3,4-tetrahydroisoquinolin-8-ol

### (A) 1′-Benzyl-6-fluoro-3H-spiro[2-benzofuran-1,4′-piperidine]

To a stirred solution of 4-fluoro-2-bromobenzoic acid (5.00 g, 22.8 mmol) in THF (15 mL) was added dropwise IM BH₃ solution in THF (52.5 mL, 52.5 mmol) at 0° C. The reaction mixture was slowly warmed up to room temperature and stirred for 24 h. The reaction mixture was quenched with 2N HCl (100 mL) and concentrated, and then the resulting residue was partitioned between ether and water. The organic layer separated was washed with brine, dried (Na₂SO₄), and concentrated. The crude product was purified by silica gel column chromatography (hexane/ethyl acetate: 4/1) to give 4.51 g (96.6%) of alcohol derivative as colorless solid. To a stirred solution of this solid (4.51 g, 22 mmol) in THF (22 mL) was added dropwise 1.56 M solution of n-butyl lithium in hexane (31 mL, 48.39 mmol) at −78° C. After 5 min stirring, the reaction mixture was slowly warmed up to 0° C. and stirred for 30 min. The reaction mixture was cooled down to −78° C. and 1-benzyl-4-piperidone (5.00 g, 26.4 mmol) was added at one portion at −78° C. The reaction mixture was warmed to room temperature and stirred for 17 h. The reaction mixture was quenched with water, concentrated, and the resulting residue was partitioned between ether and water. The organic layer separated was washed with brine, dried (Na₂SO₄), and concentrated to give orange color syrup. This was purified by amine-silica gel (CH₂Cl₂, CH₂Cl₂/methanol: 30/1) to give 2.69 g (38.8%) of the diol derivative as an orange syrup. To a stirred solution of this solid (2.69 g, 8.53 mmol) and p-toluenesulfonyl chloride (1.71 g, 8.96 mmol) in CH₂Cl₂ (9 mL) was added pyridine (9 mL) at room temperature. After 29 h stirring, the reaction mixture was partitioned between CH₂Cl₂ and water. The organic layer separated was washed with brine, dried (Na₂SO₄), and concentrated to give brown syrup. This was purified by silica gel column chromatography (CH₂Cl₂/methanol: 25/1, then hexane/ethyl acetate: 2/1) to give 350 mg (14%) of the title compound as pale yellow syrup.

¹H NMR (300 MHz, CDCl₃) δ 7.43-7.22 (5H, m), 7.13 (1H, dd, 4.8, 8.1 Hz), 6.95 (1H, ddd, 2.2, 8.5, 8.8 Hz), 6.82 (1H, dd, 2.2, 8.4 Hz), 5.02 (2H, s), 3.58 (2H, s), 2.90-2.77 (2H, m), 2.49-2.34 (2H, m), 2.02-1.87 (2H, m), 1.82-1.70 (2H, m).

MS (ESI positive) m/z: 298.12 (M+H)⁺.

### (B) 6-Fluoro-3H-spiro[2-benzofuran-1,4′-piperidine]

A mixture of 1′-benzyl-6-fluoro-3H-spiro[2-benzofuran-1,4′-piperidine] (this was prepared in the former reaction step, 350 mg, 1.18 mmol) and 1-chloroethyl chloroformate (505 mg, 3.53 mmol) in 1,2-dichloroethane (1 mL) was refluxed for 8 h. After evaporation of the solvent, the residue was diluted with methanol (4 mL) and this solution was refluxed for 3 h. After evaporation of the solvent, the residue was partitioned between ether and 1N NaOH aqueous solution. The organic layer separated was washed with brine, dried (Na₂SO₄), and concentrated to give pale yellow syrup. This was purified by silica gel column chromatography (CH₂Cl₂/methanol/triethylamine: 30/1/1) to give 178 mg (73%) of title compound as colorless solid.

¹H NMR (300 MHz, CDCl₃) δ 7.14 (1H, dd, 4.8, 8.1 Hz), 6.96 (1H, ddd, 2.2, 8.4, 8.8 Hz), 6.83 (1H, dd, 2.2, 8.4 Hz), 5.03 (2H, s), 3.15-3.00 (4H, m), 2.00-1.70 (5H, m).

MS (ESI positive) m/z: 208.02 (M+H)⁺.

### (C) 2-(tert-Butoxycarbonyl)-8-hydroxy-1,2,3,4-tetrahydroisoquinoline-3-carboxylic Acid

To a stirred suspension of 5-bromo-8-hydroxy-1,2,3,4-tetrahydroisoquinoline-3-carboxylic acid (this was prepared according to the reported method of Tetrahedron Lett., 2001, 42, 5797) in methanol (200 mL) was added thionyl chloride (9.3 mL, 127.5 mmol) dropwise at 0° C., and the reaction mixture was refluxed for 20 h. Evaporation of the solvent gave 18.71 g of white solid. To a solution of this solid (17.71 g, 54.9 mmol) and the di-tert-butyl dicarbonate (26.36 g, 121 mmol) in CH₂Cl₂ (150 mL) was added triethylamine (30.61 mL, 219.6 mmol) at 0° C., and then the reaction mixture was stirred at room temperature for 20 h. The reaction mixture was diluted with saturated aqueous solution of NaHCO₃ (300 mL), extracted with CH₂Cl₂ (300 mL×3), dried (Na₂SO₄), filtered, and concentrated. The crude product was purified by silica gel column chromatography (hexane/ethyl acetate: 4/1) to afford 26.12 g (92.6%) of 2-tert-butyl 3-methyl 8-[(tert-butoxycarbonyl)oxy]-3,4-dihydroisoquinoline-2,3(1H)-dicarboxylate as colorless syrup. A suspension mixture of this syrup (26.12 g, 53.7 mmol), 10% palladium on charcoal (1.31 g), K₂CO₃ (7.42 g, 53.7 mmol) in methanol (500 mL) was stirred under hydrogen atmosphere at room temperature for 4 h. After Celite filtration, the filtrate was concentrated to give 32.69 g of white solid. To a stirred suspension of this solid (32.69 g, 53.7 mmol) in THF (160 mL) and methanol (160 mL) was added 2N NaOH solution (80.5 mL, 161 mmol) at 0° C. and the reaction mixture was stirred at room temperature for 4 h. After evaporation of the solvent, the residue was diluted with water (200 mL), acidified with 2N HCl (75 mL) and 10% aqueous solution of citric acid (200 mL), and extracted with CH₂Cl₂ (300 mL×3). The extracts combined were dried (Na₂SO₄), filtered, and concentrated to give 15.46 g (98.2%) of the title compound as a white solid.

¹H NMR (270 MHz, DMSO-d6) δ 9.64 (1H, br.s), 6.97 (1H, dd, J=7.6, 8.1 Hz), 6.70-6.58 (2H, m), 4.95-4.89 (0.5H, m), 4.73-4.68 (0.5H, m), 4.43 (0.5H, d, J=17.3 Hz), 4.39 (0.5H, d, J=17.0 Hz), 4.28 (0.5H, d, J=17.3 Hz), 4.24 (0.5H, d, J=17.0 Hz), 3.16-2.95 (2H, m), 1.46 (4.5H, s), 1.40 (4.5H, s).

### (D) tert-Butyl 3-formyl-8-hydroxy-3,4-dihydroisoquinoline-2(1H)-carboxylate

To a stirred solution of 2-(tert-butoxycarbonyl)-8-hydroxy-1,2,3,4-tetrahydroisoquinoline-3-carboxylic acid (4.00 g, 13.6 mmol) in THF (40 mL) was added 1 M solution of borane-tetrahydrofuran complex (27.3 mL, 27.3 mmol) in THF at 0° C. After 16 h stirring at room temperature, the reaction mixture was quenched with 2N HCl (40 mL) and extracted with ethyl acetate. The extracts combined were washed with aqueous NaHCO₃ solution and brine, dried (MgSO₄), filtered, and concentrated. The residue was purified by silica gel column chromatography (hexane/ethyl acetate: 1/1) to give 3.00 g (79%) of alcohol derivative as colorless amorphous solid. To a stirred solution of this alcohol derivative (0.50 g, 1.8 mmol) and triethylamine (0.75 mL, 5.4 mmol) in DMSO (3.5 mL) was added a solution of sulfur trioxide pyridine complex (0.85 g, 5.4 mmol) in DMSO (3.5 mL) at room temperature. After 1.5 h stirring, the reaction mixture was poured into ice water and extracted with ether. The extracts combined were washed with 10% citric acid solution, water, and brine, dried (MgSO₄), filtered, and concentrated to give 0.42 g of the title compound as colorless amorphous solid. This was used for next reaction without purification.

¹H NMR (270 MHz, CDCl₃) δ 9.54 and 9.52 (total 1H, each br.s), 7.08-6.99 (1H, m), 6.78-6.60 (2H, m), 6.36 and 5.93 (total 1H, each s), 4.98-4.45 (3H, m), 3.31-3.00 (2H, m), 1.54 and 1.48 (total 9H, each s).

MS (ESI negative) m/z: 276.04 (M−H)⁻.

### (E) 3-[(6-Fluoro-1′H,3H-spiro[2-benzofuran-1,4′-piperidin]-1′-yl)methyl]-1,2,3,4-tetrahydroisoquinolin-8-ol

To a stirred solution of 6-fluoro-3H-spiro[2-benzofuran-1,4′-piperidine] (70 mg, 0.34 mmol, this was prepared in Step (B)) and tert-butyl 3-formyl-8-hydroxy-3,4-dihydroisoquinoline-2(1H)-carboxylate (122 mg, 0.44 mmol, this was prepared in Step (D)) in CH₂Cl₂ (5 mL) was added sodium triacetoxyborohydride (144 mg, 0.68 mmol) at room temperature. After 16 h stirring, the reaction mixture was quenched with aqueous NaHCO₃ solution and extracted with CH₂Cl₂. The extracts combined were dried (MgSO₄), filtered, and concentrated. The residue was purified by silica gel column chromatography (hexane/ethyl acetate: 2/1) to give 115 mg (72%) of coupling product as colorless amorphous solid. A mixture of this solid (110 mg, 0.235 mmol), trifluoroacetic acid (4 mL), and CH₂Cl₂ (1 mL) was stirred at room temperature for 3 h. After evaporation of the solvent, the residue was basified with aqueous NaHCO₃ solution and extracted with CH₂Cl₂. The extracts combined were dried (MgSO₄), filtered, and concentrated to give white solid, which was triturated with ether and collected by filtration. After washing with ether and water, the white solid was dried under vacuum to afford 58 mg (67%) of the title compound as white solid.

¹H NMR (270 MHz, CDCl₃) δ 7.17-7.10 (1H, m), 7.01-6.91 (2H, m), 6.86-6.80 (1H, m), 6.67 (1H, d, J=7.2 Hz), 6.54 (1H, d, J=7.9 Hz), 5.03 (2H, s), 4.20 (1H, d, J=15.8 Hz), 3.88 (1H, d, J=15.6 Hz), 3.11-3.00 (1H, m), 2.98-2.87 (1H, m), 2.84-2.28 (9H, m), 2.02-1.86 (2H, m), 1.82-1.71 (2H, m).

This solid (55 mg, 0.149 mmol) was converted to citric acid salt similar to that described in Example 1 to afford citric acid salt 78 mg as white solid.

MS (ESI positive) m/z: 369.21 (M+H)⁺.

IR(KBr): 1717, 1599, 1490, 1472, 1435, 1387, 1346, 1271, 1028, 781 cm⁻¹

Anal. Calcd for C₂₂H₂₅FN₂O₂—C₆H₈O₇—H₂O: C, 58.12; H, 6.10; N, 4.84. Found: C, 57.96; H, 6.12; N, 4.58.

### Example 19

### 3-[(5-Fluoro-1-methyl-1,2-dihydro-1′H-spiro[indole-3,4′-piperidin]-1′-yl)methyl]-1,2,3,4-tetrahydroisoquinolin-8-ol

### (A) Benzyl 5-fluoro-1-methyl-1,2-dihydro-1′H-spiro[indole-3,4′-piperidine]-1′-carboxylate

To a stirred suspension of 4-fluorophenylhydrazine hydrochloride (1.66 g, 10.19 mmol) in trifluoroacetic acid (2.35 mL, 30.56 mmol) and mixed solvents of acetonitrile/toluene (49/1: 20 mL) was added dropwise a solution of benzyl 4-formylpiperidine-1-carboxylate (2.29 g, 9.26 mmol) in mixed solvents of acetonitrile/toluene (49/1: 5 mL) at room temperature. After 18 h stirring at 35° C. followed by 5 h stirring at 50° C., the reaction mixture was diluted with methanol (2.5 mL) after cooling. To this reaction mixture was added NaBH₄ (525 mg, 13.89 mmol) at 0° C. After 3 h stirring at room temperature, the reaction mixture was quenched with aqueous NaHCO₃ solution (50 mL), extracted with ethyl acetate, dried (Na₂SO₄), filtered, and concentrated to give brown syrup. This crude product was purified by silica gel column chromatography (hexane/ethyl acetate: 2/1 to 1/1) to give 1.65 g (52%) of the indoline derivative as pale orange color solid. To a stirred suspension of this solid (340 mg, 1 mmol), NaBH₃CN (189 mg, 3 mmol), and 37% aqueous solution of formaldehyde (151.5 mg, 5 mmol) in methanol (10 mL) was added acetic acid (300 mg, 5 mmol) at 0° C. After 24 h stirring at room temperature, the reaction mixture was quenched with aqueous NaHCO₃ solution and extracted with ethyl acetate. The extracts combined were washed with brine, dried (Na₂SO₄), filtered, and concentrated to give an orange color syrup. This crude product was purified by silica gel column chromatography (hexane/ethyl acetate: 2/1) to give 325 mg (92%) of the title compound as pale yellow syrup.

¹H NMR (300 MHz, CDCl₃) δ 7.46-7.29 (5H, m), 6.80 (1H, ddd, J=2.6, 8.6, 9.2 Hz), 6.71 (1H, dd, J=2.6, 8.4 Hz), 6.38 (1H, dd, J=4.2, 8.5 Hz), 5.16 (2H, s), 4.25-4.05 (2H, m), 3.23 (2H, s), 3.10-2.90 (2H, m), 2.74 (3H, s), 1.85-1.65 (9H, m).

MS (ESI positive) m/z: 356.23 (M+H)⁺.

### (B) 5-Fluoro-1-methyl-1,2-dihydrospiro[indole-3,4′-piperidine]

A mixture of benzyl 5-fluoro-1-methyl-1,2-dihydro-1′H-spiro[indole-3,4′-piperidine]-1′-carboxylate (325 mg, 0.918 mmol) and 10% HCl solution in methanol (5 mL) was stirred at 50° C. for 15 h. Then the reaction mixture was refluxed for 10.5 h. After cooling, trifluoroacetic acid (3 mL) was added to the reaction mixture. After 13 h stirring at room temperature followed by 10 h stirring with reflux, the reaction mixture was concentrated to give a red oil. This was mixed with trifluoroacetic acid (5 mL) and refluxed with stirring for 2.5 h. After cooling, the reaction mixture was concentrated to give 481 mg of a pink color solid. This was partitioned between CH₂Cl₂ and aqueous NaOH solution. The organic layer separated was dried (Na₂SO₄), filtered, and concentrated to give 193 mg (96%) of a pale yellow solid.

¹H NMR (300 MHz, CDCl₃) δ 6.84-6.73 (2H, m), 6.41-6.33 (1H, m), 3.23 (2H, s), 3.12-3.00 (2H, m), 2.84-2.69 (2H, m), 2.73 (3H, s), 1.82-1.57 (5H, m).

MS (ESI positive) m/z: 221.10 (M+H)⁺.

### (C) 3-[(5-Fluoro-1-methyl-1,2-dihydro-1′H-spiro[indole-3,4′-piperidin]-1′-yl)methyl]-1,2,3,4-tetrahydroisoquinolin-8-ol

This was prepared according to the procedure described in Example 18 using 5-fluoro-1-methyl-1,2-dihydrospiro[indole-3,4′-piperidine] instead of 6-fluoro-3H-spiro[2-benzofuran-1,4′-piperidine]. Boc group was removed by treatment of 10% HCl solution in methanol instead of trifluoroacetic acid treatment. Evaporation of the solvent directly gave HCl salt of title compound as pink color solid. Overall yield was 65.1%.

MS (ESI positive) m/z: 382.21 (M+H)⁺.

IR(KBr): 3400, 1597, 1470, 1279, 1186, 1155, 1028, 1003, 941, 783 cm⁻¹

Anal. Calcd for C₂₃H₂₈FN₃O-2HCl-2.5H₂O: C, 51.55; H, 6.77; N, 7.84. Found: C, 51.69; H, 6.89; N, 7.67.

3 mg of this solid was converted to free amine.

¹H NMR (600 MHz, CDCl₃) δ 6.99 (1H, dd, J=7.7, 7.8 Hz), 6.81-6.75 (2H, m), 6.68 (1H, d, J=7.6 Hz), 6.55 (1H, d, J=7.9 Hz), 6.36 (1H, dd, J=4.1, 8.3 Hz), 4.20 (1H, d, J=15.8 Hz), 3.89 (1H, d, J=15.8 Hz), 3.20 (1H, d, J=9.1 Hz), 3.19 (1H, d, J=9.1 Hz), 3.07-3.02 (1H, m), 2.97-2.92 (1H, m), 2.83-2.78 (1H, m), 2.73 (3H, s), 2.69 (1H, dd, J=3.3, 16.0 Hz), 2.55 (1H, dd, J=10.7, 16.0 Hz), 2.55 (1H, dd, J=10.7, 16.0 Hz), 2.50 (1H, dd, J=9.6, 12.5 Hz), 2.41 (1H, dd, J=3.8, 12.4 Hz), 2.32-2.26 (1H, m), 2.11-2.04 (1H, m), 1.96-1.84 (2H, m), 1.75-1.68 (2H, m).

### Example 20

### 1′-[(8-Hydroxy-1,2,3,4-tetrahydroisoquinolin-3-yl)methyl]-1-methylspiro[indole-3,4′-piperidin]-2(1H)-one

This was prepared according to the procedure described in Example 19 using 1-methylspiro[indole-3,4′-piperidin]-2(1H)-one instead of 5-fluoro-1-methyl-1,2-dihydrospiro[indole-3,4′-piperidine]. Overall yield was 71.3%.

MS (ESI positive) m/z: 378.20 (M+H)⁺.

IR(KBr): 3400, 1686, 1612, 1595, 1493, 1472, 1383, 1279, 1094, 1016, 762 cm⁻¹

Anal. Calcd for C₂₃H₂₇N₃O₂-2HCl-0.5H₂O: C, 60.13; H, 6.58; N, 9.15. Found: C, 59.80; H, 6.59; N, 8.98.

3 mg of this solid was converted to free amine.

¹H NMR (600 MHz, CDCl₃) δ 7.46 (1H, d, J=7.3 Hz), 7.28 (1H, dd, J=7.4, 7.6 Hz), 7,06 (1H, dd, J=7.5, 7.5 Hz), 6.96 (1H, br.dd, J=7.0, 7.2 Hz), 6.85 (1H, d, J=7.7 Hz), 6.65 (1H, br.d, J=7.4 Hz), 6.58 (1H, d, J=7.9 Hz), 4.25 (1H, br.d, J=15.1 Hz), 3.92 (1H, d, J=15.1 Hz), 3.21 (3H, s), 3.17-3.07 (2H, m), 2.94-2.89 (1H, m), 2.85-2.80 (1H, m), 2.79-2.50 (5H, m), 2.05-1.80 (4H, m).

### Example 21

### 1′-[(5-Chloro-8-hydroxy-1,2,3,4-tetrahydroisoquinolin-3-yl)methyl]-1-methylspiro[indole-3,4′-piperidin]-2(1H)-one

### (A) tert-Butyl 5-chloro-3-formyl-8-hydroxy-3,4-dihydroisoquinoline-2(1H)-carboxylate

This was prepared according to the procedure described in Example 18 using 2-(tert-butoxycarbonyl)-5-chloro-8-hydroxy-1,2,3,4-tetrahydroisoquinoline-3-carboxylic acid instead of 2-(tert-butoxycarbonyl)-8-hydroxy-1,2,3,4-tetrahydroisoquinoline-3-carboxylic acid. Overall yield was 97%.

¹H NMR (300 MHz, CDCl₃) δ 9.56 (1H, s), 7.10 (1H, d, J=8.4 Hz), 6.59 (1H, d, J=8.4 Hz), 6.36 and 5.93 (total 1H, each s), 4.84-4.64 (2H, m), 4.48-4.36 (1H, m), 3.56-3.34 (1H, m), 3.05-2.90 (1H, m), 1.53 and 1.50 (total 9H, each s).

MS (ESI negative) m/z: 309.9 (M−H)⁻.

### (B) 1′-[(5-Chloro-8-hydroxy-1,2,3,4-tetrahydroisoquinolin-3-yl)methyl]-1-methylspiro[indole-3,4′-piperidin]-2(1H)-one

This was prepared according to the procedure described in Example 19 using tert-butyl 5-chloro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 59.5%.

MS (ESI positive) m/z: 412.03 (M+H)⁺.

Anal. Calcd for C₂₃H₂₆ClN₃O₂—HCl-1.5H₂O: C, 53.97; H, 6.10; N, 8.21. Found: C, 53.86; H, 5.99; N, 8.02.

This solid was converted to free amine.

¹H NMR (270 MHz, CDCl₃) δ 7.48 (1H, d, J=7.3 Hz), 7.35-7.25 (1H, m), 7.11-7.00 (2H, m), 6.87 (1H, d, J=8.1 Hz), 6.56 (1H, d, J=8.4 Hz), 4.22 (1H, br.d, J=15.9 Hz), 3.84 (1H, br.d, J=15.9 Hz), 3.22 (3H, s), 3.20-3.00 (1H, m), 3.00-2.60 (4H, m), 2.50-1.75 (8H, m).

### Example 22

### 1′-[(5-Fluoro-8-hydroxy-1,2,3,4-tetrahydroisoquinolin-3-yl)methyl]-1-methylspiro[indole-3,4′-piperidin]-2(1H)-one

This was prepared according to the procedure described in Example 19 using tert-butyl 5-fluoro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 70.3%.

MS (ESI positive) m/z: 396.07 (M+H)⁺.

Anal. Calcd for C₂₃H₂₆FN₃O₂—HCl-2H₂O: C, 54.76; H, 6.39; N, 8.33. Found: C, 55.01; H, 6.25; N, 8.13.

This solid was converted to free amine.

¹H NMR (270 MHz, CDCl₃) δ 7.48 (1H, d, J=7.1 Hz), 7.34-7.26 (1H, m), 7.08 (1H, dd, J=7.6, 7.6 Hz), 6.87 (1H, d, J=7.9 Hz), 6.71 (1H, dd, J=8.6, 9.0 Hz), 6.54 (1H, dd, J=4.9, 8.4 Hz), 4.21 (1H, br.d, J=17.0 Hz), 3.84 (1H, br.d, J=16.5 Hz), 3.22 (3H, s), 3.20-2.98 (1H, m), 2.98-2.55 (4H, m), 2.50-1.73 (8H, m).

### Example 23

### 5-Fluoro-3-[(6-fluoro-1′H,3H-spiro[2-benzofuran-1,4′-piperidin]-1′-yl)methyl]-1,2,3,4-tetrahydroisoquinolin-8-ol

This was prepared according to the procedure described in Example 19 using 6-fluoro-3H-spiro[2-benzofuran-1,4′-piperidine] instead of 1-methylspiro[indole-3,4′-piperidin]-2(1H)-one and tert-butyl 5-fluoro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 59%.

MS (ESI positive) m/z: 386.97 (M+H)⁺.

IR(KBr): 1605, 1493, 1479, 1435, 1333, 1248, 1042, 1024, 812, 737 cm⁻¹

This solid was converted to free amine.

¹H NMR (270 MHz, CDCl₃) δ 7.17-7.10 (1H, m), 7.01-6.86 (2H, m), 6.70-6.59 (1H, m), 6.54-6.45 (1H, m), 5.02 (2H, s), 4.40-4.20 (1H, m), 4.05-3.85 (1H, m), 3.30-1.95 (12H, m), 1.85-1.70 (2H, m).

### Example 24

### 5-Chloro-3-[(6-fluoro-1′H,3H-spiro[2-benzofuran-1,4′-piperidin]-1′-yl)methyl]-1,2,3,4-tetrahydroisoquinolin-8-ol

This was prepared according to the procedure described in Example 19 using 6-fluoro-3H-spiro[2-benzofuran-1,4′-piperidine] instead of 1-methylspiro[indole-3,4′-piperidin]-2(1H)-one and tert-butyl 5-chloro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 47.5%.

MS (ESI positive) m/z: 402.92 (M+H)⁺.

IR (KBr): 1618, 1456, 1296, 1041, 1024, 883, 818 cm⁻¹.

This solid was converted to free amine.

¹H NMR (270 MHz, CDCl₃) δ 7.17-7.11 (1H, m), 7.00-6.91 (3H, m), 6.52-6.48 (1H, m), 5.03 (2H, s), 4.05-3.85 (1H, m), 3.22-2.03 (12H, m), 3.00-2.60 (4H, m), 1.78-1.72 (2H, m).

### Example 25

### 5-Chloro-3-[(5-fluoro-1-methyl-1,2-dihydro-1′H-spiro[indole-3,4′-piperidin]-1′-yl)methyl]-1,2,3,4-tetrahydroisoquinolin-8-ol

This was prepared according to the procedure described in Example 19 using 5-fluoro-1-methyl-1,2-dihydrospiro[indole-3,4′-piperidine] instead of 1-methylspiro[indole-3,4′-piperidin]-2(1H)-one and tert-butyl 5-chloro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 46.2%.

MS (ESI positive) m/z: 415.90 (M+H)⁺.

IR (KBr): 1591, 1458, 1282, 1188, 1004, 941, 821 cm⁻¹.

This solid was converted to free amine.

¹H NMR (270 MHz, CDCl₃) δ 7.03 (1H, d, J=8.6 Hz), 6.82-6.75 (2H, m), 6.49 (1H, d, J=8.6 Hz), 6.40-6.35 (1H, m), 4.17 (1H, br.d, J=16.0 Hz), 3.80 (1H, br.d, J=16.0 Hz), 3.20 (2H, s), 2.86-2.76 (2H, m), 2.73 (3H, s), 2.53-2.49 (2H, m), 2.39-2.29 (2H, m), 2.10-1.70 (9H, m).

### Example 26

### 5-Fluoro-3-[(5-fluoro-1-methyl-1,2-dihydro-1′H-spiro[indole-3,4′-piperidin]-1′-yl)methyl]-1,2,3,4-tetrahydroisoquinolin-8-ol

This was prepared according to the procedure described in Example 19 using 5-fluoro-1-methyl-1,2-dihydrospiro[indole-3,4′-piperidine] instead of 1-methylspiro[indole-3,4′-piperidin]-2(1H)-one and tert-butyl 5-fluoro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 21.2%.

MS (ESI positive) m/z: 399.93 (M+H)⁺.

IR (KBr): 1605, 1475, 1248, 1186, 1010, 822, 737 cm⁻¹.

This solid was converted to free amine.

¹H NMR (270 MHz, CDCl₃) δ 6.82-6.67 (3H, m), 6.52-6.47 (1H, m), 6.40-6.35 (1H, m), 4.17 (1H, br.d, J=16.2 Hz), 3.83 (1H, br.d, J=16.2 Hz), 3.20 (2H, s), 2.99-2.73 (2H, m), 2.73 (3H, s), 2.53-2.44 (2H, m), 2.37-2.27 (2H, m), 2.12-1.70 (9H, m).

### Example 27

### 1′-[(5-Chloro-8-hydroxy-1,2,3,4-tetrahydroisoquinolin-3-yl)methyl]-5-fluoro-1-methylspiro[indole-3,4′-piperidin]-2(1H)-one

This was prepared according to the procedure described in Example 19 using 5-fluoro-1-methylspiro[indole-3,4′-piperidin]-2(1H)-one instead of 1-methylspiro[indole-3,4′-piperidin]-2(1H)-one and tert-butyl 5-chloro-3-formyl-8-hydroxy-3,4-dihydroisoquinoline-2(1H)-carboxylate instead of tert-butyl 3-formyl-8-hydroxy-3,4-dihydroisoquinoline-2(1H)-carboxylate. Overall yield was 21.2%.

MS (ESI positive) m/z: 430.02 (M+H)⁺.

IR (KBr): 1693, 1622, 1591, 1458, 1364, 1273, 1167, 1005, 947, 866, 816, 704 cm⁻¹.

This solid was converted to free amine.

¹H NMR (270 MHz, CDCl₃) δ 7.24-7.20 (1H, m), 7.04-6.96 (2H, m), 6.80-6.76 (1H, m), 6.56-6.53 (1H, m), 4.19 (1H, br.d, J=15.8 Hz), 3.80 (1H, br.d, J=15.8 Hz), 3.21 (3H, s), 3.15-2.27 (11H, m), 2.07-1.96 (2H, m), 1.82-1.72 (2H, m).

### Example 28

### 1′-[(5-Chloro-1,2,3,4-tetrahydroisoquinolin-3-yl)methyl]-1-methyl-1,2-dihydrospiro[indole-3,4′-piperidine]

### (A) 5-Chloro-1-oxo-1,2,3,4-tetrahydroisoquinoline-3-carboxylic acid

To a stirred suspension of 3-chloro-2-methylbenzoic acid (10.00 g, 58.6 mmol) in methanol (100 mL) was added dropwise thionyl chloride (8.5 mL, 117.2 mmol) at 0° C. The reaction mixture was refluxed for 2 h. After cooling down to room temperature, the reaction mixture was concentrated in vacuo. The residue was dissolved in ethyl acetate (300 mL), washed with 1N NaOH solution (100 mL), water (100 mL) and brine (100 mL), dried (Na₂SO₄), filtered, and concentrated to give 10.86 g of methyl ester as colorless oil. A mixture of this ester (10.80 g, 58.5 mmol), N-bromosuccinimide (10.45 g, 64.4 mmol), and benzoyl peroxide (0.71 g, 2.9 mmol) in carbon tetrachloride (70 mL) was refluxed for 3 h. After cooling down to room temperature, the reaction mixture was filtered and the filtrate wad concentrated to give 16.30 g of methyl 3-chloro-2-bromomethylbenzoate as a colorless oil. To a stirred suspension of NaH (2.45 g, 61.3 mmol) in DMF (50 mL) was added dropwise a solution of diethyl acetamidomalonate (12.10 g, 55.7 mmol) in DMF (50 mL) at room temperature. After 15 min stirreing, a solution of methyl 3-chloro-2-bromomethylbenzoate (16.3 g, 58.5 mmol) in DMF (50 mL) at room temperature. After 18 h stirring, the reaction mixture was concentrated. The residue was dissolved in ethyl acetate (800 mL), washed with water (200 mL×2) and brine (200 mL), dried (Na₂SO₄), filtered, and concentrated to give 21.5 g of yellow solid, which was purified by silica gel column chromatography (hexane/ethyl acetate: 2/1 to 1/1) to afford 14.33 g (64.3%) of coupling product as a white solid. A mixture of this solid (13.90 g, 34.7 mmol) and 47% HBr (160 mL) was refluxed for 24 h. After cooling down to room temperature, the solid formed was collected by filtration and dried togive 6.16 g (78.6%) of title compound as a cream color solid.

¹H NMR (300 MHz, DMSO-d6) δ 8.26 (1H, d, J=4.0 Hz), 7.85 (1H, dd, J=1.1, 7.7 Hz), 7.64 (1H, dd, J=1.3, 8.1 Hz), 7.39 (1H, dd, J=7.9, 7.9 Hz), 4.34-4.28 (1H, m), 3.42-3.36 (1H, overlapped with water peak at 3.36 ppm), 3.26 (1H, dd, J=6.8, 16.9 Hz).

### (B) 1′-[(5-Chloro-1,2,3,4-tetrahydroisoquinolin-3-yl)methyl]-1-methyl-1,2-dihydrospiro[indole-3,4′-piperidine]

A mixture of 5-chloro-1-oxo-1,2,3,4-tetrahydroisoquinoline-3-carboxylic acid (150 mg, 0.66 mmol), 1-methyl-1,2-dihydrospiro[indole-3,4′-piperidine] (165 mg, 0.60 mmol), WSC (127 mg, 0.66 mmol), 1-hydroxybenzotriazole (89 mg, 0.66 mmol), and triethylamine (0.09 mL, 0.66 mmol) in DMF (5 mL) was stirred at room temperature for 4 h. The reaction mixture was diluted with water (50 mL), basified with 2N NaOH, and extracted with ethyl acetate (150 mL). The extract was washed with brine (30 mL), dried (Na₂SO₄), filtered, and concentrated to give 650 mg of yellow oil, which was purified by silica gel column chromatography (CH₂Cl₂/methanol/NH₄OH: 400/10/1) to afford 234 mg (95%) of amide derivative as a colorless gum. A mixture of this amide derivative (230 mg, 0.56 mmol) and borane-methyl sulfide complex (0.27 mL, 2.8 mmol) in THF (20 mL) was refluxed for 20 h. After cooling down to room temperature, 6N HCl was added to the reaction mixture. After 5 h stirring with reflux, the reaction mixture was cooled down to room temperature, basified with 2N NaOH, and extracted with ethyl acetate (100 mL). The extract was washed with brine (30 mL), dried (Na₂SO₄), filtered, and concentrated to give 290 mg of a colorless oil, which was purified by silica gel column chromatography (CH₂Cl₂/methanol/NH₄OH: 200/10/1) to afford 162.6 mg (77%) of title compound as a pale yellow oil.

¹H NMR (300 MHz, CDCl₃) δ 7.23-7.19 (1H, m), 7.13 (1H, dd, J=1.3, 7.5 Hz), 7.11-7.05 (3H, m), 6.72 (1H, ddd, J=0.9, 7.3, 7.5 Hz), 6.49 (1H, d, J=7.7 Hz), 4.09 (2H, br.s), 3.25-2.79 (6H, m), 2.78 (3H, s), 2.60-2.28 (4H, m), 2.14-1.66 (6H, m).

This solid was converted to citric acid salt similar to that described in Example 1.

MS (ESI positive) m/z: 382.0 (M+H)⁺.

IR(KBr): 1720, 1670, 1568, 1340, 1215, 1134, 758 cm⁻¹

### Example 29

### 5-Chloro-3-(2,3-dihydro-1′H-spiro[indene-1,4′-piperidin]-1′-ylmethyl)-3,4-dihydroisoquinolin-[(2H)-one

To a stirred suspension of 5-chloro-1-oxo-1,2,3,4-tetrahydroisoquinoline-3-carboxylic acid (500 mg, 2.22 mmol) in THF (10 mL) was added borane-methyl sulfide complex (0.53 m]L, 5.55 mmol) at 0° C. After 40 h stirring at room temperature, the reaction mixture was diluted with water (20 mL) and 2N HCl (20 mL) and extracted with ethyl acetate (200 mL). The extract was washed with brine (40 mL), dried (Na₂SO₄), filtered, and concentrated to give 336.4 mg of colorless gum. This was purified by silica gel column chromatography (CH₂Cl₂/methanol: 20/1) to afford 193.5 mg (41.2%) of alcohol derivative as a white solid. To a stirred solution of this solid (190 mg, 0.90 mmol) and triethylamine (0.38 mL, 2.7 mmol) in DMSO (2.5 mL) was added sulfur trioxide pyridine complex (430 mg, 2.7 mmol) in DMSO (2.5 mL) at room temperature. After 1 h stirring, the reaction mixture was poured into ice water (100 mL) and extracted with ethyl acetate (100 mL). The extract was washed with aqueous 10% citric acid solution (20 mL) and water (20 mL), dried (Na₂SO₄), filtered, and concentrated to give 160 mg of aldehyde derivative. To a stirred suspension of this aldehyde derivative (160 mg) and 2,3-dihydrospiro[indene-1,4′-piperidine] (70 mg, 0.37 mmol) in THF (5 mL) was added sodium triacetoxyborohydride (157 mg, 0.74 mmol) at room temperature. After 72 h stirring, the reaction mixture was diluted with water (10 mL), basified with saturated aqueous solution of NaHCO₃, and extracted with CH₂Cl₂ (80 mL×2). The extracts combined were dried (Na₂SO₄), filtered, and concentrated to give 178 mg of yellow solid, which was purified by silica gel column chromatography (hexane/ethyl acetate: 2/1) to afford 52.8 mg (37.5%) of title compound as a white solid.

¹H NMR (300 MHz, CDCl₃) δ 8.04 (1H, d, J=7.7 Hz), 7.52(1H, d, J=8.1 Hz), 7.31 (1H, dd, J=7.7, 8.0 Hz), 7.25-7.16 (4H, m), 6.84 (1H, br.s), 3.94-3.82 (1H, m), 3.22 (1H, dd, J=3.7, 15.9 Hz), 2.90-2.74 (4H, m), 2.66-2.42 (4H, m), 2.17-1.83 (5H, m), 1.60-1.50 (2H, m).

This solid was converted to citric acid salt similar to that described in Example 1.

MS (ESI positive) m/z: 381.0 (M+H)⁺.

IR(KBr): 3400, 1719, 1607, 1493, 1450, 1383, 1190, 959, 754 cm⁻¹

Anal. Calcd for C₂₃H₂₅ClN₂O—C₆H₈O₇-2H₂O: C, 57.19; H, 6.12; N, 4.60. Found: C, 57.36; H, 5.74; N, 4.35.

### Example 30

### 3-[(5-Fluoro-1-methyl-1,2-dihydro-1′H-spiro[indole-3,4′-piperidin]-1′-yl)methyl]-8-hydroxy-3,4-dihydro-1H-isochromen-1-one

### (A) 3-[(5-Fluoro-1-methyl-1,2-dihydro-1′H-spiro[indole-3,4′-piperidin]-′-yl)methyl]-8-methoxy-3,4-dihydro-1H-isochromen-1-one

To a solution of 3-(iodomethyl)-8-methoxy-3,4-dihydro-1H-isochromen-1-one (this was prepared according to the reported methods in J. Org. Chem. 1996, 61 (13), 4190-4191 and Tetrahedron, 2002, 58, 6455; 146.3 mg, 0.46 mmol) in DMF (8.0 μL) was added 5-fluoro-1-methyl-1,2-dihydrospiro[indole-3,4′-piperidine] (101.3 mg, 0.46 mmol) followed by diisopropylethylamine (0.321 mL, 1.84 mmol) at r.t. under N₂. The mixture was stirred at r.t. for 3 h, then warmed to 90° C. in a sealed-tube, stirred for 3 days, and then, allowed to r.t., diluted with Et2O (20 mL), poured into H₂O (50 mL), extracted with Et₂O (40 mL×3). The ethereal layers were combined, dried over MgSO₄, concentrated in vacuo, the residue was purified by silica gel PLC (CH₂Cl₂/MeOH: 30/1) to afford 115.6 mg (61.4%) of title compound as a slight brown solid.

¹H NMR (300 MHz, CDCl₃) 7.47 (1H, t, J=7.5 Hz), 6.92 (1H, d, J=8.3 Hz), 6.86-6.75 (4H, m), 6.38-6.33 (1H, m), 4.59 (1H, m), 3.95 (3H, s), 3.20 (2H, s), 3.00-2.67 (6H, m), 2.73 (3H, s), 2.39-2.21 (2H, m), 1.93-1.67 (4H, m).

MS (ESI positive) m/z: 411 (M+H)⁺.

### (B) 3-[(5-Fluoro-1-methyl-1,2-dihydro-1′H-spiro[indole-3,4′-piperidin]-1′-yl)methyl]-8-hydroxy-3,4-dihydro-1H-isochromen-1-one

To a solution of 3-[(5-fluoro-1-methyl-1,2-dihydro-1′H-spiro[indole-3,4′-piperidin]-1′-yl)methyl]-8-methoxy-3,4-dihydro-1H-isochromen-1-one (115 mg, 0.28 mmol) (prepared in the former reaction step) in CH₂Cl₂ (10 mL) was added 1 M solution of borone tribromide (0.98 mL, 0.98 mmol) in CH₂Cl₂ at 0° C. under N₂. The mixture was stirred at 0° C. for 2 h, quenched with aqueous NaHCO₃ solution (10 mL), extracted with ethyl acetate, dried (Na₂SO₄), filtered, and concentrated to give brown solid. This crude product was purified by silica gel column chromatography (hexane/ethyl acetate: 1/1) to give 78.9 mg (71%) of title compound as a white solid.

¹H NMR (300 MHz, CDCl₃) δ 10.90 (1H, br.s), 7.44 (1H, t, J=8.4 Hz), 6.91 (1H, d, J=8.1 Hz), 6.83-6.73 (3H, m), 6.40-6.35 (1H, m), 4.93 (1H, br.s), 3.21 (2H, s), 3.08-2.91 (5H, m), 2.73 (3H, s), 2.55-1.60 (7H, m).

This was converted to HCl salt similar to that described in Example 2 to afford 29.5 mg of HCl salt.

MS (ESI positive) m/z: 397 (M+H)⁺.

Anal. Calcd for C₂₃H₂₅FN₂O₃-2HCl-3H₂O: C, 52.78; H, 6.35; N, 5.35. Found: C, 52.82; H, 6.10; N, 5.04.

### Example 31

### 3-(2,3-Dihydro-1′H-spiro[indene-1,4′-piperidin]-1′-ylmethyl)-3,4-dihydro-1H-isochromen-8-ol

### (A) 3-(2,3-Dihydro-1′H-spiro[indene-1,4′-piperidin]-1′-ylmethyl)-8-methoxy-3,4-dihydro-1H-isochromen-1-one

To a solution of 3-(iodomethyl)-8-methoxy-3,4-dihydro-1H-isochromen-1-one (prepared in Example 30) in THF was added 2,3-dihydrospiro[1H-indene-1,4′-piperidine] hydrochloride (202.6 mg, 0.91 mmol) followed by triethylamine (0.505 mL, 3.6 mmol) at r.t. under N₂. The mixture was stirred at r.t. for 3 h, then warmed to 60° C., stirred for 30 min, and then stirred under reflux condition for 38 h, and then allowed to r.t., poured into saturated NaHCO₃ aq. (70 mL), extracted with Et₂O (70 mL×3). The ethereal layers were combined, dried over MgSO₄, concentrated in vacuo, the residue was purified by silica gel column chromatography (CH₂Cl₂/MeOH/25% NH₃aq.: 35/1/0.15) to afford 84.7 mg (25%) of the title compound as colorless oil.

¹H NMR (300 MHz, CDCl₃) δ 7.46 (1H, t, J=7.5 Hz), 7.23-7.13 (5H, m), 6.92 (2H, d, J=8.4 Hz), 6.86 (2H, dd, J=7.5 Hz, 0.75 Hz), 4.61 (1H, m), 3.95 (3H, s), 3.01-2.68 (8H, m), 2.46-2.28 (2H, m), 2.04-1.86 (2H, m), 1.55-1.49 (2H, m).

MS (ESI positive) m/z: 378 (M+H)⁺.

### (B) 1-(2,3-Dihydro-1′H-spiro[indene-1,4′-piperidin]-1′-yl)-3-[2-(hydroxymethyl)-3-methoxyphenyl]propan-2-ol

To a stirred solution of 3-(2,3-dihydro-1′H-spiro[indene-1,4′-piperidin]-1′-ylmethyl)-8-methoxy-3,4-dihydro-1H-isochromen-1-one (58.7 mg, 0.16 mmol) in THF (4 mL) was added LiAlH₄ (27 mg, 0.71 mmol) at room temperature under N₂. After stirring for 2 h at room temperature, the reaction mixture was quenched with MeOH (4 drops) and water (8 drops) at 0° C. After 5 min stirring, water (10 mL) was added to the reaction mixture and stirring was continued another 15 min. The mixture was poured into saturated NaHCO₃, extracted with (8 mL×4). The extracts combined were dried (MgSO₄), filtered, and concentrated to afford 63.7 mg of crude title compound as viscosity oil.

¹H NMR (300 MHz, CDCl₃) δ 7.27-7.12 (5H, m), 6.81 (2H, d, J=8.0 Hz), 4.87 (1H, d, 11.7 Hz), 4.64 (1H, d, 11.7 Hz), 3.86 (3H, s), 3.75 (1H, m), 2.99-2.73 (6H, m), 2.58-1.48 (10H, m).

MS (ESI positive) m/z: 382 (M+H)⁺.

### (C) 1′-[(8-Methoxy-3,4-dihydro-1H-isochromen-3-yl)methyl]-2,3-dihydrospiro[indene-1,4′-piperidine]

This procedure was partially according to the reported method (Tetrahedron Lett., 1998, 39, 6751).

(Step a) A mixture of TsOH—H₂O (30 mg) and SiO₂ (Wakogel C-300HGT, 180 mg) in CHCl₃ (4 mL) was stirred at 15-30° C. for 30 min with heating by handy-drier, and then the solvent was removed in vacuo.

(Step b) To a stirred solution of 1-(2,3-dihydro-1′H-spiro[indene-1,4′-piperidin]-1′-yl)-3-[2-(hydroxymethyl)-3-methoxyphenyl]propan-2-ol (40 mg) in CHCl₃ (6 mL) was added the above TsOH—H₂O/SiO₂ (ca. 105 mg) at a time at r.t., stirred for 1.5 h, then more TsOH—H₂O/SiO₂ (ca. 105 mg) was added, stirred for 19 h at r.t., then warmed to 30-50° C. for 30 min. A solution of CHCl₃ (20 mL)-MeOH (2 mL)-25% NH₃aq. (0.5 mL) was added to the above mixture, stirred for 30 min, and then filtered with CHCl₃-MeOH-25% NH₃aq. (ca.100 mL). The eluent was concentrated in vacuo, and the residue was purified by PLC (SiO₂, CH₂Cl₂/MeOH: 25/1) and then (CH₂Cl₂/MeOH/25% NH₃aq.: 35/1/0.1) to afford 8.2 mg of a title product.

¹H NMR (300 MHz, CDCl₃) δ 7.24-7.12 (5H, m), 6.74 (1H, d, J=7.3 Hz), 6.68 (1H, d, J=8.0 Hz), 4.98 (1H, d, 16 Hz), 4.66 (1H, d, 16 Hz), 3.92 (1H, m), 3.80 (3H, s), 3.15-1.53 (16H, m).

MS (ESI positive) m/z: 364 (M+H)⁺.

### (D) 3-(2,3-Dihydro-1′H-spiro[indene-1,4′-piperidin]-1′-ylmethyl)-3,4-dihydro-1H-isochromen-8-ol

To a solution of 1′-[(8-methoxy-3,4-dihydro-1H-isochromen-3-yl)methyl]-2,3-dihydrospiro[indene-1,4′-piperidine] (8.2 mg) in DMF (0.7 mL) was added EtSNa (60 mg 0.71 mmol) at r.t. and then stirred at 120° C. under N₂ for 17 h and 150° C. for 3 h, allowed to r.t., then, concentrated in vacuo. The residue was partitioned between water (9 mL) and CHCl₃(14 mL), adjusted to pH 7.5-8 by adding saturated NH₄Claq (several drops) with stirring. The organic layer was separated and the aqueous layer was extracted with CHCl₃ (14 mL×2). The organicl layers were combined, dried over MgSO₄, concentrated in vacuo, the residue was purified by silica gel PLC (CH₂Cl₂/MeOH/25% NH₃aq.: 20/1/0.1) to afford 3.4 mg (61.4%) of title compound.

¹H NMR (300 MHz, CDCl₃) δ 7.24-7.13 (4H, m), 7.04 (1H, t, J=7.9 Hz), 6.72 (1H, d, J=7.5 Hz), 6.57 (1H, d, J=7.9 Hz), 5.3 (1H, brs), 5.00 (1H, d, 16 Hz), 4.70 (1H, d, 16 Hz), 3.93 (1H, m), 3.02 (2H, m), 2.90 (2H, t, 7.3 Hz), 2.80-1.54 (12H, m).

MS (ESI positive) m/z: 350 (M+H)⁺.

### Pharmaceutical Composition Examples

In the following Examples, the term ‘active compound’ or ‘active ingredient’ refers to a compound of formula (I) or a pharmaceutically acceptable salt, solvate or pro-drug thereof, according to the present invention.

(i) Tablet Compositions

The following compositions A and B can be prepared by wet granulation of ingredients (a) to (c) and (a) to (d) with a solution of povidone, followed by addition of the magnesium stearate and compression.

Composition A

| | mg/tablet | mg/tablet |
|---|---|---|
| (a) Active ingredient | 250 | 250 |
| (b) Lactose B.P. | 210 | 26 |
| (c) Sodium Starch Glycollate | 20 | 12 |
| (d) Povidone B.P. | 15 | 9 |
| (e) Magnesium Stearate | 5 | 3 |
| | 500 | 300 |

Composition B

| | mg/tablet | mg/tablet |
|---|---|---|
| (a) Active ingredient | 250 | 250 |
| (b) Lactose 150 | 150 | — |
| (c) Avicel PH 101 | 60 | 26 |
| (d) Sodium Starch Glycollate | 20 | 12 |
| (e) Povidone B.P. | 15 | 9 |
| (f) Magnesium Stearate | 5 | 3 |
| | 500 | 300 |

Composition C

| | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in formulation E is of the direct compression type.

Composition D

| | mg/tablet |
|---|---|
| Active ingredient | 250 |
| Magnesium Stearate | 4 |
| Pregelatinised Starch NF15 | 146 |
| | 400 |

Composition E

| | mg/tablet |
|---|---|
| Active ingredient | 250 |
| Magnesium Stearate | 5 |
| Lactose | 145 |
| Avicel | 100 |
| | 500 |

Composition F (Controlled release composition)

| | mg/tablet |
|---|---|
| (a) Active ingredient | 500 |
| (b) Hydroxypropylmethylcellulose | 112 |
| (Methocel K4M Premium) | |
| (c) Lactose B.P. | 53 |
| (d) Povidone B.P.C. | 28 |
| (e) Magnesium Stearate | 7 |
| | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

Composition B

| | mg/capsule |
|---|---|
| (a) Active ingredient | 250 |
| (b) Lactose B.P. | 143 |
| (c) Sodium Starch Glycollate | 25 |
| (d) Magnesium Stearate | 2 |
| | 420 |

Composition C

| | mg/capsule |
|---|---|
| (a) Active ingredient | 250 |
| (b) Macrogol 4000 BP | 350 |
| | 600 |

Capsules can be prepared by melting the Macrogol 4000 BP, dispersing the active ingredient in the melt and filling two-part hard gelatin capsules therewith.

Composition D

| | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled release capsule)

| | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)

| | mg/capsule |
|---|---|
| (a) Active ingredient | 250 |
| (b) Microcrystalline Cellulose | 125 |
| (c) Lactose BP | 125 |
| (d) Cellulose Acetate Phthalate | 50 |
| (e) Diethyl Phthalat | 5 |
| | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

(iii) Intravenous injection composition

| | |
|---|---|
| Active ingredient | 0.200 g |
| Sterile, pyrogen-free phosphate buffer (pH 9.0) to | 10 ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | |
|---|---|
| Active ingredient | 0.20 g |
| Benzyl Alcohol | 0.10 g |
| Glycofurol 75 | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | |
|---|---|
| Active ingredient | 0.25 g |
| Sorbitol Solution | 1.50 g |
| Glycerol | 1.00 g |
| Sodium Benzoate | 0.005 g |
| Flavour | 0.0125 ml |
| Purified Water q.s. to | 5.0 ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | |
|---|---|
| Active ingredient | 200 mg |
| Alcohol USP | 0.1 ml |
| Hydroxyethyl cellulose | |

The active ingredient and alcohol USP are gelled with hydroxyethyl cellulose and packed in a transdermal device with a surface area of 10 cm².

1. A compound of the following formula (I) 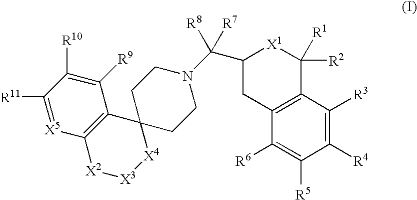
or a pharmaceutically acceptable ester or amide of such compound, or a pharmaceutically acceptable salt thereof, wherein
X¹ represents
an oxygen atom; or
N—R¹² wherein R¹² is selected from the group consisting of a hydrogen atom, an alkyl group having 1 to 6 carbon atoms, an alkanoyl group having 1 to 6 carbon atoms, an alkylaminocarbonyl group having 1 to 6 carbon atoms in the alkyl group, an aryl group as defined below, an aryalkyl group having 1 to 6 carbon atoms in the alkyl part and the aryl part as defined below, a heteroaryl group as defined below and a heteroarylalkyl group having 1 to 6 carbon atoms in the alkyl part and heteroaryl part as defined below;
R¹ and R² each independently represent a hydrogen atom;
an alkyl group having 1 to 6 carbon atoms;
an alkoxy group having 1 to 6 carbon atoms;
an alkanoyl group having 1 to 6 carbon atoms;
an alkylcarbonylamino group having 1 to 6 carbon atoms in the alkyl part;
an alkylaminocarbonyl group having 1 to 6 carbon atoms in the alkyl part;
a mono-hydroxyalkyl group having 1 to 6 carbon atoms;
a mono-aminoalkyl having 1 to 6 carbon atoms; or
an alkoxyalkyl group having 1 to 6 carbon atoms in the alkoxy group and 1 to 6 carbon atoms in the alkyl part; or R¹ and R² taken together form oxo;
R³, R⁴, R⁵ and R⁶ each independently represent
a hydrogen atom;
a halogen atom;
a hydroxy group;
an alkyl group having 1 to 6 carbon atoms;
an alkoxy group having 1 to 6 carbon atoms;
an alkanoyl group having 1 to 6 carbon atoms;
a mono-hydroxyalkyl group having 1 to 6 carbon atoms;
a mono-aminoalkyl group having 1 to 6 carbon atoms;
an alkylcarbonylamino group having 1 to 6 carbon atoms in the alkyl part;
an alkylaminocarbonyl group having 1 to 6 carbon atoms in the alkyl part;
an alkylaminosulfonyl group having 1 to 6 carbon atoms in the alkyl part;
an aryl group as defined below which is linked directly to the benzene ring or is attached via a spacer group to the benzene ring, and the spacer group is defined as below; or
a heteroaryl group as defined below which is linked directly to the benzene ring or is attached via a spacer group to the benzene ring, and the spacer group is defined as below;
provided that at least one of R³ through R⁶ must represents a hydrogen atom;
R⁷ and R⁸ both represent hydrogen atoms or taken together form oxo;
R⁹, R¹⁰ and R¹¹ each independently represent a hydrogen atom, a halogen atom or an alkyl group having 1 to 6 carbon atoms;
X², X³ and X⁴ each independently represent methylene, an oxygen atom, NR¹³, where R¹³ is defined as a hydrogen atom or an alkyl group having from 1 to 6 carbon atoms, or carbonyl, provided that at least one of X², X³ and X⁴ must represent methylene or carbonyl; or
X⁴ represents a bond and X² and X³ each independently represent methylene, an oxygen atom, NR¹³, where R¹³ is defined as a hydrogen atom or an alkyl group having from 1 to 6 carbon atoms, or carbonyl, provided that at least one of X² and X³ must be methylene or carbonyl;
wherein the methylene in the definitions of X², X³ and X⁴ is each independently unsubstituted or substituted by at least one alkyl groups having 1 to 6 carbon atoms;
X⁵ represents a —CR¹⁴ or a nitrogen atom wherein R¹⁴ represents a hydrogen atom, a halogen atom or an alkyl group having 1 to 6 carbon atoms;
said amino parts of the alkylcarbonylamino groups and alkylaminocarbonyl groups in the definitions of R¹ through R⁶ and R¹² are unsubstituted or substituted by an alkyl group having 1 to 6 carbon atoms;
said aryl groups and aryl parts of aralkyl groups referred to in the definitions of R¹ through R⁶ and R¹² are aromatic hydrocarbon groups having 5 to 14 carbon atoms;
said heteroaryl groups and heteroaryl parts of the heteroarylalkyl groups referred to in the definitions of R³ through R⁶ and R¹² are 5- to 7-membered heteroaryl groups containing 1 to 3 oxygen, sulfur and/or nitrogen atoms; and
said spacer groups referred to in the definitions of R¹ and R² are each independently selected from the groups consisting of an oxygen atom, sulfonyl and carbonyl.

2. The compound according to claim 1, wherein X¹ represents N—R¹² and R¹² represents a hydrogen atom, an alkyl group having 1 to 6 carbon atoms or an alkanoyl group having 1 to 6 carbon atoms.

3. The compound according to claim 1, wherein X¹ represents an oxygen atom.

4. The compound according to claim 1, wherein X⁴ represents a bond and X² and X³ each independently represent methylene, an oxygen atom, NR¹³, where R¹³ is defined as a hydrogen atom or an alkyl group having from 1 to 6 carbon atoms, or carbonyl, provided that at least one of X² and X³ must be methylene or carbonyl, wherein the methylene is unsubstituted or is substituted by at least one alkyl group having 1 to 6 carbon atoms.

5. The compound according to claim 1, wherein X⁵ represents CR¹⁴.

6. The compound according to claim 1, wherein R⁷ and R⁹ both represent hydrogen atoms.

7. The compound according to claim 1, wherein R¹ and R² both represent hydrogen atoms or one of R¹ and R² is a hydrogen atom and the other one is an alkyl group having 1 to 6 carbon atoms.

8. The compound according to claim 1, wherein R¹ and R² taken together form oxo.

9. The compound according to claim 1, wherein R³ to R each independently represent a hydrogen atom, a hydroxy group or a halogen atom, provided that at least one of R³ to R⁶ must represent a hydrogen atom.

10. The compound according to claim 1 selected from
1′-(1,2,3,4-tetrahydroisoquinolin-3-ylmethyl)-2,3-dihydrospiro[indene-1,4′-piperidine];
1′-[(2-acetyl-1,2,3,4-tetrahydroisoquinolin-3-yl)methyl]-2,3-dihydrospiro[indene-1,4′-piperidine];
1′-(2-methyl-1,2,3,4-tetrahydroisoquinolin-3-yl)methyl]-2,3-dihydrospiro[indene-1,4′-piperidine];
1′-[(3S)-1,2,3,4-tetrahydroisoquinolin-3-ylmethyl]-2,3-dihydrospiro[indene-1,4′-piperidine];
(3R)-3-(2,3-dihydro-1′H-spiro[indene-1,4′-piperidin]-1′-ylmethyl)-1,2,3,4-tetrahydroisoquinolin-7-ol;
3-(2,3-dihydro-1′H-spiro[indene-1,4′-piperidin]-1′-ylmethyl)-1,2,3,4-tetrahydroisoquinolin-6-ol;
5-bromo-3-(2,3-dihydro-1′H-spiro[indene-1,4′-piperidin]-1′-ylmethyl)-1,2,3,4-tetrahydroisoquinolin-8-ol;
3-(2,3-dihydro-1′H-spiro[indene-1,4′-piperidin]-1′-ylmethyl)-2-methyl-1,2,3,4-tetrahydroisoquinolin-8-ol;
3-(2,3-dihydro-1′H-spiro[indene-1,4′-piperidin]-1′-ylmethyl)-1,2,3,4-tetrahydroisoquinolin-8-ol;
3-(2,3-dihydro-1′H-spiro[indene-1,4′-piperidin]-1′-ylmethyl)-5-fluoro-1,2,3,4-tetrahydroisoquinolin-8-ol
5-fluoro-3-[(1-methyl-1,2-dihydro-1′H-spiro[indole-3,4′-piperidin]-1′-yl)methyl]-1,2,3,4-tetrahydroisoquinolin-8-ol;
5-chloro-3-(2,3-dihydro-1′H-spiro[indene-1,4′-piperidin]-1′-ylmethyl)-1,2,3,4-tetrahydroisoquinolin-8-ol;
5-chloro-3-(1′H,3H-spiro[2-benzofuran-1,4′-piperidin]-1′-ylmethyl)-1,2,3,4-tetrahydroisoquinolin-8-ol;
5-chloro-3-[(1-methyl-1,2-dihydro-1′H-spiro [indole-3,4′-piperidin]-1′-yl)methyl]-1,2,3,4-tetrahydroisoquinolin-8-ol;
1′-{[(3S)-1-methyl-1,2,3,4-tetrahydroisoquinolin-3-yl]methyl}-2,3-dihydrospiro[indene-1,4′-piperidine];
3-[(3,3-dimethyl-1′H,3H-spiro[2-benzofuran-1,4′-piperidin]-1′-yl)methyl]-1,2,3,4-tetrahydroisoquinolin-8-ol;
3-[(1′-methyl-1′,2′-dihydro-1H-spiro[piperidine-4,3′-pyrrolo[2,3-b]pyridin]-1-yl)methyl]-1,2,3,4-tetrahydroisoquinolin-8-ol;
3-[(6-fluoro-1′H,3H-spiro[2-benzofuran-1,4′-piperidin]-1′-yl)methyl]-1,2,3,4-tetrahydroisoquinolin-8-ol;
3-[(5-fluoro-1-methyl-1,2-dihydro-1′H-spiro[indole-3,4′-piperidin]-1′-yl)methyl]-1,2,3,4-tetrahydroisoquinolin-8-ol;
1′-[(8-hydroxy-1,2,3,4-tetrahydroisoquinolin-3-yl)methyl]-1-methylspiro[indole-3,4′-piperidin]-2(1H)-one;
1′-[(5-chloro-8-hydroxy-1,2,3,4-tetrahydroisoquinolin-3-yl)methyl]-1-methylspiro[indole-3,4′-piperidin]-2(1H)-one;
1′-[(5-fluoro-8-hydroxy-1,2,3,4-tetrahydroisoquinolin-3-yl)methyl]-1-methylspiro[indole-3,4′-piperidin]-2(1H)-one;
5-fluoro-3-[(6-fluoro-1′H,3H-spiro[2-benzofuran-1,4′-pi piperidin]-1′-yl)methyl]-1,2,3,4-tetrahydroisoquinolin-8-ol;
5-chloro-3-[(6-fluoro-1′H,3H-spiro[2-benzofuran-1,4′-piperidin]-1′-yl)methyl]-1,2,3,4-tetrahydroisoquinolin-8-ol;
5-chloro-3-[(5-fluoro-1-methyl-1,2-dihydro-1′H-spiro[indole-3,4′-piperidin]-1′-yl)methyl]-1,2,3,4-tetrahydroisoquinolin-8-ol;
5-fluoro-3-[(5-fluoro-1-methyl-1,2-dihydro-1′H-spiro[indole-3,4′-piperidin]-1′-yl)methyl]-1,2,3,4-tetrahydroisoquinolin-8-ol;
1′-[(5-chloro-8-hydroxy-1,2,3,4-tetrahydroisoquinolin-3-yl)methyl]-5-fluoro-1-methylspiro[indole-3,4′-piperidin]-2(1H)-one;
1′-[(5-chloro-1,2,3,4-tetrahydroisoquinolin-3-yl)methyl]-1-methyl-1,2-dihydrospiro[indole-3,4′-piperidine] and
5-chloro-3-(2,3-dihydro-1′H-spiro[indene-1,4′-piperidin]-1′-ylmethyl)-3,4-dihydroisoquinolin-1(2H)-one or a pharmaceutically acceptable salt thereof.

11. The compound according to claim 1 selected from 3-[(5-fluoro-1-methyl-1,2-dihydro-1′H-spiro[indole-3,4′-piperidin]-1′-yl)methyl]-8-hydroxy-3,4-dihydro-1H-isochromen-1-one; and 3-(2,3-dihydro-1′H-spiro[indene-1,4′-piperidin]-1′-ylmethyl)-3,4-dihydro-1H-isochromen-8-ol or a pharmaceutically salt thereof.

12. A pharmaceutical composition comprising a compound according to claim 1 and pharmaceutically acceptable excipient, diluent or carrier.

13. A method of treating a disease or condition for which an ORL1 antagonist is indicated, in a mammal comprising administering to the mammal requiring such treatment an effective amount of a compound of claim 1 or a pharmaceutically acceptable salt, solvate or composition thereof.

14. The method according to claim 13 wherein the disease or condition is selected from pain; sleep disorders, eating disorders including anorexia and bulimia; anxiety and stress conditions; immune system diseases; locomotor disorder; memory loss, cognitive disorders and dementia including senile dementia, Alzheimer's disease, Parkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding properties of drug of abuse, modulation of striatal and glutamate effects on locomotor activity; cardiovascular disorders including hypotension, bradycardia and stroke; renal disorders including water excretion, sodium ion excretion and syndrome of inappropriate secretion of antidiuretic hormone (SIADH); gastrointestinal disorders; airway disorders including adult respiratory distress syndrome (ARDS); autonomic disorders including suppression of micturition reflex; metabolic disorders including obesity; cirrhosis with ascites; sexual dysfunctions; altered pulmonary function including obstructive pulmonary disease; and tolerance to or dependency on a narcotic analgesic.

15. A method according to claim 13 wherein the disease or condition is pain, sleep disorders, eating disorders including anorexia and bulimia; stress conditions; memory loss, cognitive disorders, gastrointestinal disorders; sexual dysfunctions; and tolerance to or dependency on a narcotic analgesic.
